(19) United States
(12) Patent Application Publication
(10) Pub. No.: US 2026/0354432 A1
(43) Pub. Date: Nov. 12, 2026

(54) HYDRATION COMPOSITION

(71) Applicant: Conopco, Inc., d/b/a UNILEVER, Hoboken, NJ (US)

(72) Inventors: Sean Paul LAVIN, Los Angeles, CA (US); Alexandra Matti Helene NORRED, Los Angeles, CA (US)

(73) Assignee: Conopco, Inc., d/b/a UNILEVER, Hoboken, NJ (US)

(21) Appl. No.: 19/102,036

(22) PCT Filed: Aug. 11, 2023
(86) PCT No.: PCT/EP2023/072316

§ 371 Date: Feb. 7, 2025

Related U.S. Application Data
(60) Provisional application No. 63/397,302, filed on Aug. 11, 2022.

(30) Foreign Application Priority Data
Aug. 31, 2022 (EP) 22193060.5

(51) Int. Cl. A23L 33/16; A23L 2/38; A23L 2/56; A23L 2/60; A23L 33/15; A23L 33/175; A61K 9/00; A61K 31/198; A61K 31/375; A61K 31/4415; A61K 31/7004; A61K 31/714; A61K 33/14; A61K 47/02

(52) U.S. Cl. CPC A23L 33/16; A23L 2/38; A23L 2/56; A23L 2/60; A23L 33/15; A23L 33/175; A61K 9/0095; A61K 31/198; A61K 31/375; A61K 31/4415; A61K 31/7004; A61K 31/714; A61K 33/14; A61K 47/02

(57) ABSTRACT

A hydration composition comprising: allulose; one or more sodium salts; and one or more potassium salts; wherein the composition has less than about 10%, preferably less than about 5%, more preferably less than about 3%, and most preferably less than about 1%, or 0%, by weight, of a nutritive or artificial sweetener; and wherein, optionally, at least one of the one or more sodium salts and/or one or more of the potassium salts is a citrate salt, preferably sodium citrate, disodium citrate, trisodium citrate, potassium citrate and/or mixtures or combinations thereof.

### FIELD OF THE INVENTION

The present invention is directed to a hydration composition comprising allulose, one or more sodium salts, and one or more potassium salts, and having less than about 10%, preferably less than about 5%, more preferably less than about 3%, and most preferably less than about 1%, or 0%, by weight, of a nutritive or artificial sweetener, as well as formulations and methods of making and using the hydration composition.

### BACKGROUND OF THE INVENTION

Hydration formulas generally relate to beverages, or pre-mixes thereof, that are designed to help prevent dehydration. Dehydration can lead to severe and serious health complications including but not limited to seizures, kidney failure, and in some cases, may even be life threatening. However, being sufficiently hydrated will help avoid such serious health complications, and helps regulate body temperature, prevent infections, deliver nutrients to cells, and keeps organs functioning properly.

It is believed that some known hydration formulations exploit the glucose-sodium active transport mechanism to enhance absorption of water into the bloodstream. In the small intestine, sodium passes into intestinal epithelial cells via co-transport with glucose by way of the SGLT1 protein. Two sodium ions and one molecule of glucose are transported together across the epithelial cell membrane to the bloodstream via the SGLT1 protein. The absorption of glucose and sodium out of the small intestine creates an osmotic force. For each transport cycle, water molecules move into the epithelial cells to maintain osmotic equilibrium. Water that is absorbed from the lumen of the small intestine rather than being excreted is available for systemic distribution by the bloodstream for cellular hydration. Water is thus delivered to the bloodstream faster and in larger quantities than by drinking water alone.

Yet, many consumers desire a hydration formula that does not contain sugar, such as glucose, for many reasons including health concerns (such as diabetes) as well as a desire to reduce sugar intake and/or have a glucose sensitivity. It is expected that consumers would also prefer a hydration formula with little or no artificial sweetener.

Accordingly, the inventors of the present application desired to create an oral hydration or replacement solution without including glucose, and without a significant amount of nutritive or artificial sugars, while still delivering faster and better hydration than water alone, as well as providing a desirable taste for consumers. Additionally, the inventors of the present application desired to create an oral hydration formula to meet the requirements of the World Health Organization (WHO) osmolality ranges 210-268 mOsm/L without including glucose, and without a significant amount of nutritive or artificial sugars, while still delivering faster and better hydration than water alone.

### SUMMARY OF THE INVENTION

In a first aspect, provided is a hydration composition comprising allulose; one or more sodium salts; and one or more potassium salts; wherein the composition has less than less than about 10%, preferably less than about 5%, more preferably less than about 3%, and most preferably less than about 1%, or 0%, by weight, of a nutritive or artificial sweetener; and wherein, optionally, at least one of the one or more sodium salts and/or one or more of the potassium salts is a citrate salt, preferably sodium citrate, disodium citrate, trisodium citrate, potassium citrate and/or mixtures or combinations thereof.

In a second aspect, provided is a hydration formulation comprising the hydration composition as described herein.

In another aspect, provided is a hydration formulation comprising a hydration composition, wherein the hydration composition comprises: (a) core base, wherein the core base comprises allulose and sodium chloride; and (b) an electrolyte base, wherein the electrolyte base comprises one or more potassium salts, and one or more sodium salt excludes sodium chloride; wherein, optionally, the core base comprises at least one amino acid, and wherein, optionally, at least one of the one or more sodium salts and/or one or more of the potassium salts is a citrate salt, preferably sodium citrate, disodium citrate, trisodium citrate, potassium citrate and/or mixtures or combinations thereof.

In yet another aspect, provided is a method for making the hydration composition as described herein.

Additionally, results from studies reported below show that while the movement of water across the epithelia is, at least partially, dependent on the presence of an osmotic gradient, correcting the osmotic gradient favored water absorption induced by an allulose-containing hydration formula with limited amounts of stevia and otherwise free of nutritive, non-nutritive and artificial sweeteners. Moreover, the highest concentration of the allulose-containing test formula (C3) induced the water absorption from luminal to basolateral compartment even in the presence of higher osmolality in the luminal compartment. This result suggests that the allulose-containing hydration formula with limited amounts of stevia and otherwise free of nutritive, non-nutritive and artificial sweeteners is capable of transporting water against the osmotic gradient. Since transport via the SGLT1 protein involves glucose and the test product contains allulose rather than glucose, this is an interesting finding.

Another set of experiments reported below were performed after middle/long term exposure to allulose-containing hydration formula with limited amounts of stevia and otherwise free of nutritive, non-nutritive and artificial sweeteners, followed by a recovery of 6 hrs in the presence of mannitol in the lower compartment, to investigate a potential effect of an allulose-containing hydration formula with limited amounts of stevia and otherwise free of nutritive, non-nutritive and artificial sweeteners (SF) on mannitol-mediated water absorption. SF increased the TEER values after 48 hrs, suggesting it might increase the barrier tightness, which could be an additional unexpected beneficial effect of the test product. Overall, SF was shown to promote intestinal water absorption in this model as well as increase epithelial barrier tightness.

All other aspects of the present invention will more readily become apparent from the description and examples which follow.

### BRIEF DESCRIPTION OF THE DRAWINGS

FIG. 1 presents graphs showing the effect of a hydration formula with glucose (HM) and a test solution, SF (an allulose-containing hydration formula with limited amounts of stevia and otherwise free of nutritive, non-nutritive and artificial sweeteners on transepithelial electrical resistance (TEER) (A, C, E, and G) and liquid absorption (B, D, F, and H) in Caco-2/TC7 monolayers. TEER and liquid volume were measured after 1, 3, 6, and 24 hours of treatment. TEER values represent % in comparison to the values obtained before the treatments. Liquid volume are reported as mL, positive values represent passage from luminal to basolateral compartment, while negative values represent passage from basolateral to luminal compartment. Results are Mean±SD of 1 experiment, in triplicate.

FIG. 2 shows the calculated osmolarity at the luminal and basolateral compartments, according to the treatments described below.

FIG. 3. Graphs showing the effect of a glucose-containing rehydration formula (HM) and allulose-containing hydration formula with limited amounts of stevia and otherwise free of nutritive, non-nutritive and artificial sweeteners (SF) on transepithelial electrical resistance (TEER) (A, C) and liquid absorption (B, D) in Caco-2/TC7 monolayers. TEER values represent % in comparison to the values obtained before the treatments. Liquid volume are reported as mL, positive values represent passage from luminal to basolateral compartment, while negative values represent passage from basolateral to luminal compartment. Results are Mean±SD of 1 experiment, in triplicate.

FIG. 4. Graphs showing effect of treatments on cell viability (A) and relative paracellular permeability (B). Cell viability was assessed by MTT assay and it represents % in comparison to Control. Relative paracellular permeability represents the % of passage of permeability tracker LY across Caco-2/TC7 monolayers. Results are Mean±SD of 1 experiment, in triplicate, and are at 24 hours.

FIG. 5. Effect of treatments on cell viability (A) and relative paracellular permeability (B). Cell viability was assessed by MTT assay and it represents % in comparison to Control. Relative paracellular permeability represents the % of passage of permeability tracker LY across Caco-2/TC7 monolayers. Experiments were performed after 24 hours of treatment followed by 6 hours of exposure to mannitol 2.5% in the lower compartment (recovery time). Results are Mean±SD of 1 experiment, in triplicate.

FIG. 6. Graphs showing the effect of treatments on cell viability (A) and relative paracellular permeability (B). Cell viability was assessed by MTT assay and it represents % in comparison to Control. Relative paracellular permeability represents the % of passage of permeability tracker LY across Caco-2/TC7 monolayers. Experiments were performed after 48 hours of treatment followed by 6 hours of exposure to mannitol 2.5% in the lower compartment (recovery time). Results are Mean±SD of 1 experiment, in triplicate.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of any of the compositions or components thereof, unless otherwise specified. It should be noted that in specifying any ranges of values, any particular upper value can be associated with any particular lower value.

For the avoidance of doubt, the term “comprising” is meant not to be limiting to any stated elements but rather to encompass non-specified elements of major or minor functional importance. Therefore, the listed steps, elements or options need not be exhaustive.

Whenever the words “including” or “having” are used, these terms are meant to be equivalent to “comprising” as defined above.

The disclosure as found herein, is to be considered to cover all embodiments as found in the claims as being multiply dependent upon each other irrespective of the fact that claims may be found without explicit multiple dependency or redundancy.

### Hydration Composition

A hydration composition as used herein, includes at least: allulose, one or more sodium salts and one or more potassium salts.

In one aspect, provided is a hydration composition comprising: (a) allulose; (b) one or more sodium salts; and (c) one or more potassium salts, wherein the hydration composition has less than about 10%, preferably less than about 5%, more preferably less than about 3%, and most preferably less than about 1%, or 0%, by weight, of any of nutritive or artificial sweetener. In one aspect, the hydration composition is in the form of a powder, gel, emulsion, or effervescent tablet, having less than about 5%, or less than about 3%, or than about 1%, or about 0%, or about 0% to about 1%, or about 0.005% to about 0.5%, or about 0.01% to about 0.1% by weight water. In one aspect, at least one amino acid is included in the hydration composition.

In one aspect, provided is a hydration composition comprising: (a) a core base comprising allulose and sodium chloride, and optionally at least one amino acid, and (b) an electrolyte base comprising one or more potassium salts, and optionally, one or more sodium salts other than sodium chloride, wherein the hydration composition has less than about 10%, preferably less than about 5%, more preferably less than about 3%, and most preferably less than about 1%, or 0%, by weight, of any of nutritive or artificial sweetener. As used herein, the “core base” and “electrolyte base” are precursors to the hydration composition. In one aspect, the hydration composition is in the form of a powder, gel, emulsion, or effervescent tablet, having less than about 5%, or less than about 3%, or than about 1%, or about 0%, or about 0% to about 1%, or about 0.005% to about 0.5%, or about 0.01% to 0.1% by weight water.

In one aspect, the hydration composition includes at least one citrate salt. As described herein, a citrate salt refers to sodium citrates such as disodium citrate and trisodium citrate, potassium citrate, and zinc citrate. In one aspect, at least one of the one or more sodium salts and/or one or more of the potassium salts is a citrate salt, preferably sodium citrate, disodium citrate, trisodium citrate, potassium citrate and/or mixtures or combinations thereof.

As used herein, allulose, which also refers to d-allulose, psicose, and/or d-psicose, refers to a rare sugar which has significantly less calories than sucrose. In an embodiment, the amount of allulose in the hydration composition as described herein, is about 20% to about 80%, or about 20% to about 40%, or about 25% to about 35%, or about 28% to about 32%, or about 50% to about 80%, or about 50% to about 70%, or about 55% to about 70%, or about 55% to about 65%, or about 58% to about 63%, based on the total weight of the hydration composition, including any amount or ranges subsumed therein.

As used herein, the term “nutritive or artificial sweetener” refers to glucose, fructose, galactose, sucrose, lactose, maltose, dextrose, sucralose, honey, agave, molasses, corn syrup (including high fructose corn syrup), xylitol, sorbitol, acesulfame potassium (Ace-K), monk fruit (Luo Han Guo), sodium saccharide, saccharin, erythritol, neotame, tagatose, aspartame, and stevia such as RebA, Reb D, RebM, and steviol glycosides. As used herein, a nutritive or artificial sweetener does not include allulose. The hydration composition as described herein, has less than about 10%, preferably less than about 5%, more preferably less than about 3%, and most preferably less than about 1%, or 0%, by weight, based on the total weight of the hydration composition, of a nutritive or artificial sweetener. In one aspect, the hydration composition as described herein, is free of any nutritive or artificial sweetener. In one aspect, the hydration composition as described herein, has about 0.01% to about 10%, or about 0.01% to about 5%, or 0.01% to about 3%, or about 0.01% to about 2%, or about 0.01 to about 1.5%, or about 0.01 to about 1.2%, or about 0.01 to about 1.1% or about 0.01 to about 1% or about 0.01 to about 0.9%, or about 0.01 to about 0.5%, or about 0.1% to about 3%, or about 0.1% to about 2%, or about 0.1 to about 1.5%, or about 0.1 to about 1.2%, or about 0.1 to about 1.1% or about 0.1 to about 1% or about 0.1 to about 0.9%, or about 0.1 to about 0.5%, based on the total weight of the hydration composition, including any amount or ranges subsumed therein. In one aspect, the nutritive or artificial sweetener is stevia, in the absence of any other nutritive or artificial sweeteners, as defined herein.

In one aspect, the one or more sodium salts included in the hydration composition is sodium chloride, monosodium phosphate, disodium phosphate, trisodium phosphate, sodium citrates (such as disodium citrate and/or trisodium citrate), sodium carbonate, sodium bicarbonate, sodium lactate, sodium gluconate, tetrasodium acid pyrophosphate, sodium acid sulfate, sodium hydroxide, and/or mixtures or combination thereof. In one aspect the sodium salt is sodium chloride, sodium citrates and/or mixtures or combinations thereof.

In one aspect, the total amount of the one more sodium salts is about 5% to about 20%, or about 5% to about 15%, or about 5% to about 12%, or about 7% to about 18%, or about 7% to about 15%, or about 7% to about 12%, or about 8% to about 12%, or about 9% to about 12%, based on the total weight of the hydration composition, including any amounts or ranges subsumed therein.

In one aspect, sodium chloride may be at least one of the one or more sodium salts included the hydration composition. In one aspect, the amount of sodium chloride is about 2% to about 10%, or about 4% to about 10%, or about 5% to about 10%, or about 6% to about 10%, or about 7% to about 10%, or about 4% to about 8%, or about 5% to about 8%, or about 6% to about 8%, based on the total weight of the hydration composition, including any amounts or ranges subsumed therein.

In one aspect, sodium citrates, such as disodium citrate and trisodium citrate, may be at least one of the one or more sodium salts included in the hydration composition. In one aspect, the amount of sodium citrates, such as disodium citrate and/or trisodium citrate is about 1% to about 10%, or about 1% to about 8%, or about 1% to about 6%, or about 1% to about 5%, or about 2% to about 8%, or about 2% to about 6%, or about 2% to about 5%, or about 3% to about 5%, based on the total weight of the hydration composition, including any amounts or ranges subsumed therein. In one aspect, the weight ratio of allulose to sodium citrates in the hydration composition as described herein, is about 20:1 to about 5:1, or about 20:1 to about 15:1, or about 18:1 to about 16:1, or about 10:1 to about 5:1, or about 9:1 to about 6:1, or about 8:1 to about 6:1.

In one aspect, the one or more sodium salts are sodium chloride and sodium citrates. In one aspect, the weight ratio of sodium chloride to sodium citrates in the hydration composition as described herein, is about 3:1 to about 1:3, or about 3:1 to about 1:1, or about 2:1 to about 1:2, or about 2:1 to about 1:1.

In one aspect, the one or more potassium salts included in the hydration composition is potassium chloride, potassium phosphate, dipotassium phosphate, potassium citrate, potassium carbonate, potassium bicarbonate, potassium hydroxide, and/or mixtures or combination thereof. In one aspect, the one or more potassium salts is dipotassium phosphate, potassium citrate, and/or mixtures or combination thereof.

In one aspect, the total amount of the one or more potassium salts is about 1% to about 20%, or about 3% to about 15%, or about 3% to about 12% or about 3% to about 10%, or about 3% to about 8%, or about 5% to about 12%, or about 5% to about 10%, or about 5% to about 8%, based on the total weight of the hydration composition, including any amounts or ranges subsumed therein.

In one aspect, potassium citrate may be at least one of the one or more potassium salts included in the hydration composition included in the hydration composition. In one aspect, the amount of potassium citrate is about 1% to about 10%, or about 1% to about 8%, or about 3% to about 9%, or about 3% to about 7%, or about 3% to about 6%, or about 4% to about 6%, based on the total weight of the hydration composition, including any amounts or ranges subsumed therein. In one aspect, the weight ratio of allulose to potassium citrate in the hydration composition as described herein, is about 20:1 to about 5:1, or about 18:1 to about 10:1, or about 15:1 to about 10:1, or about 15:1 to about 12:1.

In one aspect, dipotassium phosphate may be at least one of the one or more potassium salts included in the hydration composition. In one aspect, the amount of dipotassium phosphate is about 1% to about 10%, or about 1% to about 5%, or about 1% to about 3%, or about 1.5% to about 3%, or about 1.5% to about 2.5%, based on the total weight of the hydration composition, including any amounts or ranges subsumed therein.

In one aspect, the one or more potassium salts are potassium citrate and dipotassium phosphate. In one aspect, the weight ratio of potassium citrate to dipotassium phosphate in the hydration composition as described herein, is about 4:1 to about 1:4, or about 4:1 to about 2:1, or about 4:1 to about 1:1, or about 3:1 to about 1:3, or about 3:1 to about 1:1.

In one aspect, the total amount of sodium salt(s) and potassium salt(s) in the hydration composition as described herein, is about 5% to about 30%, or about 10% to about 30%, or about 10% to about 20%, or about 15% to about 30%, or about 15% to about 20% based on the total weight of the hydration composition, including any amounts or ranges subsumed therein. In one aspect, the weight ratio of allulose to total amount of sodium salt(s) and potassium salt(s) in the hydration composition as described herein, is about 5:1 to about 1:1, or about 5:1 to about 1.5:1, or about 5:1 to about 2:1, or about 5:1 to about 3:1, or about 4:1 to 1:1, or about 4:1 to about 1.5:1, or about 4:1 to about 2:1, or about 4:1 to about 3:1, or about 2:1 to about 1:2, or about 2:1 to about 1:1. In one aspect, the one or more sodium salts are sodium chloride and/or sodium citrates, and the one or more potassium salts are dipotassium phosphate and/or potassium citrate. In one aspect, the weight ratio of allulose to total amount of citrate salts in the hydration composition as described herein, is about 10:1 to 1:1, or about 10:1 to about 5:1, or about 10:1 to about 7:1, or about 9:1 to about 6:1, or about 9:1 to about 7:1, or about 4:1 to 1:1, or about 4:1 to about 2:1, or about 4:1 to about 3:1, or about 2:1 to about 1:2, or about 2:1 to about 1:1. In one aspect, the citrate salts are sodium citrate and/or potassium citrate.

In one aspect, the hydration composition optionally contains calcium chloride, calcium carbonate, magnesium chloride, magnesium carbonate, or a combination thereof, in an amount of about 0.1% to about 10%, or about 0.1% to about 5%, or about 0.1% to about 2%, or about 0.1% to about 1.5%, or about 0.1 to about 1%, or about 0.5% to about 2%, or about 0.5% to about 1.5%, or about 0.5% to about 1%, or about 1% to about 10%, or about 1% to about 5%, or about 1% to about 3%, or about 1.5% to about 3%, or about 1.5% to about 2.5%, based on the total weight of the hydration composition, including any amounts or ranges subsumed therein.

In one aspect, the hydration composition as described herein, optionally contains at least one amino acid or an amino acid analogue. In one aspect, the amino acid(s) included in the hydration composition may be sodium glutamate, potassium glutamate, L-glutamic acid monosodium hydrate, D-glutamic acid monosodium hydrate, L-glutamic acid, D-glutamic acid, D-alanine, D-glutamine, lysine, L-lysine, threonine, L-threonine, serine, L-serine, leucine, L-leucine, iso-leucine, valine, L-valine and/or any salts, combinations or mixtures thereof. In one aspect, the hydration composition includes at least one of glutamine, L-glutamine, alanine, L-alanine, glycine, L-glycine, alanyl-glutamine, L-alanyl-glutamine and/or any salts, combinations, or mixtures thereof.

In one aspect, at least one amino acid is included in the hydration composition as described herein, in an amount of about 1% to about 15%, or about 5% to about 15%, or about 8% to about 15%, or about 10% to about 15%, or about 12% to about 15%, based on the total weight of the hydration composition, including any amounts or ranges subsumed therein. In one aspect, glutamine or L-glutamine is included in the hydration composition as described herein, in an amount of about 1% to about 15%, or about 5% to about 15%, or about 8% to about 15%, or about 10% to about 15%, or about 12% to about 15%, based on the total weight of the hydration composition, including any amounts or ranges subsumed therein. In one aspect, alanine or L-alanine is included in the hydration composition as described herein, in an amount of about 1% to about 15%, or about 5% to about 15%, or about 8% to about 15%, or about 10% to about 15%, or about 12% to about 15%, based on the total weight of the hydration composition, including any amounts or ranges subsumed therein. In one aspect, alanyl-glutamine or L-alanyl-glutamine is included in the hydration composition as described herein, in an amount of about 1% to about 15%, or about 5% to about 15%, or about 8% to about 15%, or about 10% to about 15%, or about 12% to about 15%, based on the total weight of the hydration composition, including any amounts or ranges subsumed therein.

In one aspect, the total amount of amino acids included in the hydration composition as described herein, is about 1% to about 30%, or about 1% to about 15%, or about 1% to about 10%, or about 5% to about 30%, or about 5% to about 15%, or about 10% to about 30%, or about 15% to about 30%, or about 20% to about 30%, or about 25% to about 30%, based on the total weight of the composition, including any amounts or ranges subsumed therein. In one aspect, the amino acids are L-glutamine, L-alanine, alanyl-glutamine, L-alanyl-glutamine, or mixtures or combinations thereof. In one aspect, the amino acids are L-glutamine and/or L-alanine.

In one aspect, the weight ratio of allulose to total amino acid in the hydration composition as described herein, is about 3:1 to about 1:1, or about 2:1 to about 1:2, or about 2:1 to about 1:1.

In one aspect, the hydration composition as described herein, may optionally include at least one processing aid. A processing aid may be silicon dioxide, sodium aluminum silicate, magnesium stearate, rice hull powder, and any combination thereof. In one aspect, the total amount of processing aids included in the hydration composition, as described herein, is about 0.01% to about 5%, or about 0.01% to about 2%, or about 0.1% to about 5%, or about 0.1% to about 3%, or about or about 0.1% to about 2%, or about or about 0.1% to about 1.5%, or about 0.5% to about 1.5%, based on the total weight of the hydration composition, including any amounts or ranges subsumed therein.

The hydration composition as described herein, may optionally contain additional agent(s). Additional agents, as used herein, refers to, for example, vitamins, minerals, caffeine, proteins, herbal/botanical/plant supplements, nutraceuticals, enzymes, probiotics, prebiotics, postbiotics, fermented ingredients, polysaccharides, fats, phytosterols, soluble fiber, insoluble fiber, antioxidants, glucosamine, glycosaminoglycans, adaptogens, nootropics, hormones, flavoring, colorants, preservatives, anti-caking agents, gelling agents, antimicrobials, citric acid and/or other acidifiers, emulsifiers, effervescing agents, and/or excipients. In one aspect, the total amount of additional agent(s) present in the hydration composition as disclosed herein is about 0.001% to about 30%, or about 0.01% to about 30%, about 0.1% to about 30%, or 0.001% to about 20%, or about 0.01% to about 20%, about 0.1% to about 20%, or about 0.001% to about 15%, or about 0.01% to about 15%, or about 0.01% to about 10%, or about 0.01% to about 5%, or about 0.01% to about 2%, or about 0.01% to about 1%, about or about 0.1 to about 10%, or about 0.1 to about 5%, or about 0.1 to about 2%, or about 0.1 to about 1%, or about 0.1 to about 0.5%, based on the total weight of the composition, including any amounts or ranges subsumed therein. In one aspect, each additional agent may be present in an amount of about 0.001% to about 15%, or about 0.01% to about 15%, or about 0.01% to about 10%, or about 0.01% to about 5%, or about 0.01% to about 2%, or about 0.01% to about 1%, about or about 0.1 to about 10%, or about 0.1 to about 5%, or about 0.1 to about 2%, or about 0.1 to about 1%, or about 0.1 to about 0.5%, based on the total weight of the composition, including any amounts or ranges subsumed therein.

In one aspect, vitamins may optionally be included in the hydration composition, including but not limited to vitamin A, vitamin B1, vitamin B2, vitamin B3, vitamin B5, vitamin B6, vitamin B7, vitamin B12, vitamin C, vitamin D, vitamin E, vitamin K, and any combinations thereof. In one aspect, the hydration composition includes vitamin B and vitamin C, preferably vitamin B3, vitamin B5, vitamin B6, vitamin B12, and vitamin C.

In one aspect, one or more mineral may optionally be included, generally salts, in the hydration composition, including but not limited to zinc, copper, manganese, nickel, tin, silicon, molybdenum, selenium, chromium, vanadium, boron, calcium, iron, iodine, phosphorus, and any combinations thereof.

In one aspect, herbal/botanical/plant supplements may optionally be included in the hydration composition, including but not limited to valerian, chamomile, passionflower, yerba mate, ginger, carrot, echinacea, mushrooms (such as reishi or cordyceps), coconut (such as coconut water and coconut water) ginseng, ashwagandha, theobromine, and any combinations thereof.

In one aspect, antioxidants may optionally be included in the hydration composition, including but not limited to carotene, eugenol, lutein, lycopene, EGCG, green tea extract, white tea, green coffee extract, and any combinations thereof.

In one aspect, a probiotic and/or a prebiotics may optionally be included in the hydration composition, including but not limited to Lactobacillus reuteri, Lactobacillus acidophilus, and Bifidobacterium bifidum, Bacillus coagulans, inulin, FOS, GOS, XOS, bacteriophages.

In one aspect, a fermented ingredients may optionally be included in the hydration composition, including but not limited to fermented ginger, fermented lemongrass, short chain fatty acids, acetic acid, lactic acid, butyric acid, and propionic acid.

In one aspect, a hormone may optionally be included in the hydration composition, including but not limited to melatonin.

In one aspect, a flavoring may optionally be included in the hydration composition. Flavorings, as described herein, may have about 0.01% to about 2%, or about 0.01% to about 1.5%, or about 0.01 to about 1.2%, or about 0.01 to about 1.1%, or about 0.01% to about 1%, or about 0.01% to about 0.5%, or about 0.01% to about 0.25%, or about 0.01% to about 0.1%, or 0% of a nutritive or artificial sweetener as described herein, by weight of the flavoring composition.

In one aspect, the hydration composition as described herein, in the form of a powder, gel, emulsion, or effervescent tablet, having about 0% to about 1%, or about 0.005% to about 0.5%, or about 0.01% to about 0.1% by weight water, includes (a) allulose in an amount of about 50% to about 80%, or about 50% to about 70%, or about 55% to about 70%, or about 55% to about 65%, or about 58% to about 63%, based on the total weight of the hydration composition; (b) one or more sodium salts, in an amount of about 7% to about 15%, or about 7% to about 12%, or about 8% to about 12%, or about 9% to about 12%, based on the total weight of the hydration composition; and (c) one or more potassium salts, in an amount of about 3% to about 10%, or about 3% to about 8%, or about 5% to about 12%, or about 5% to about 10%, or about 5% to about 8%, based on the total weight of the hydration composition. In one aspect, the hydration composition has at least one citrate salt selected from sodium citrates, potassium citrate, zinc citrate, and/or any combinations or mixtures thereof. In one aspect, at least one of the one or more sodium salts and/or one or more of the potassium salts is a citrate salt, preferably sodium citrate, disodium citrate, trisodium citrate, potassium citrate and/or mixtures or combinations thereof. In one aspect, the amount of a nutritive or artificial sweetener is about 0.01% to about 3%, or about 0.01% to about 2%, or about 0.01% to about 1.5%, or about 0.01% to about 1.2%, or about 0.01% to about 1.1% or about 0.01% to about 1%, or 0% based on the total weight of the hydration, wherein nutritive or artificial sweetener is stevia. In one aspect, the hydration composition further comprises a processing aid in an amount of about 0.01% to about 5%, or about 0.01% to about 2%, or about 0.1% to about 5%, or about 0.1% to about 3%, or about 0.1% to about 2%, or about 0.1% to about 1.5%, or about 0.5% to about 1.5%, based on the total weight of the hydration composition, including any amounts or ranges subsumed therein. In one aspect, at least one additional agent is included in the hydration composition selected from vitamins, minerals, caffeine, proteins, herbal/botanical/plant supplements, nutraceuticals, enzymes, probiotics, prebiotics, postbiotics, fermented ingredients, polysaccharides, fats, phytosterols, soluble fiber, insoluble fiber, antioxidants, glucosamine, glycosaminoglycans, adaptogens, nootropics, hormones, flavoring, colorants, preservatives, anti-caking agents, gelling agents, antimicrobials, citric acid and/or other acidifiers, emulsifiers, effervescing agents, and/or excipients.

In one aspect, the hydration composition as described herein, in the form of a powder, gel, emulsion, or effervescent tablet, having about 0% to about 1%, or about 0.005% to about 0.5%, or about 0.01% to about 0.1% by weight water, includes (a) allulose in an amount of about 50% to about 80%, or about 50% to about 70%, or about 55% to about 70%, or about 55% to about 65%, or about 58% to about 63%, based on the total weight of the hydration composition; (b) one or more sodium salts, wherein the one or sodium salts is sodium chloride, sodium citrate and/or a mixture or combination thereof in an amount of about 7% to about 15%, or about 7% to about 12%, or about 8% to about 12%, or about 9% to about 12%, based on the total weight of the hydration composition; and (c) one or more potassium salts, wherein the potassium salt is potassium citrate, dipotassium phosphate or mixtures thereof in an amount of about 3% to about 10%, or about 3% to about 8%, or about 5% to about 12%, or about 5% to about 10%, or about 5% to about 8%, based on the total weight of the hydration composition. In one aspect, the amount of a nutritive or artificial sweetener is about 0.01% to about 3%, or about 0.01% to about 2%, or about 0.01% to about 1.5%, or about 0.01% to about 1.2%, or about 0.01% to about 1.1% or about 0.01% to about 1%, or 0% based on the total weight of the hydration, wherein nutritive or artificial sweetener is stevia. In one aspect, the hydration composition further comprises a processing aid in an amount of about 0.01% to about 5%, or about 0.01% to about 2%, or about 0.1% to about 5%, or about 0.1% to about 3%, or about 0.1% to about 2%, or about 0.1% to about 1.5%, or about 0.5% to about 1.5%, based on the total weight of the hydration composition, including any amounts or ranges subsumed therein. In one aspect, at least one additional agent is included in the hydration composition selected from vitamins, minerals, caffeine, proteins, herbal/botanical/plant supplements, nutraceuticals, enzymes, probiotics, prebiotics, postbiotics, fermented ingredients, polysaccharides, fats, phytosterols, soluble fiber, insoluble fiber, antioxidants, glucosamine, glycosaminoglycans, adaptogens, nootropics, hormones, flavoring, colorants, preservatives, anti-caking agents, gelling agents, antimicrobials, citric acid and/or other acidifiers, emulsifiers, effervescing agents, and/or excipients.

In one aspect, the hydration composition as described herein, in the form of a powder, gel, emulsion, or effervescent tablet, having about 0% to about 1%, or about 0.005% to about 0.5%, or about 0.01% to about 0.1% by weight water, includes (a) allulose in an amount of about 20% to about 40%, or about 25% to about 35%, or about 28% to about 32%, based on the total weight of the hydration composition; (b) one or more sodium salts, in an amount of about 7% to about 15%, or about 7% to about 12%, or about 8% to about 12%, or about 9% to about 12%, based on the total weight of the hydration composition; (c) one or more potassium salts, in an amount of about 3% to about 10%, or about 3% to about 8%, or about 5% to about 12%, or about 5% to about 10%, or about 5% to about 8%, based on the total weight of the hydration composition. In one aspect, the hydration composition has at least one citrate salt selected from sodium citrates, potassium citrate, zinc citrate, and/or any combinations or mixtures thereof. In one aspect, at least one of the one or more sodium salts and/or one or more of the potassium salts is a citrate salt, preferably sodium citrate, disodium citrate, trisodium citrate, potassium citrate and/or mixtures or combinations thereof. In one aspect, the amount of a nutritive or artificial sweetener is about 0.01% to about 3%, or about 0.01% to about 2%, or about 0.01% to about 1.5%, or about 0.01% to about 1.2%, or about 0.01% to about 1.1% or about 0.01% to about 1%, or 0% based on the total weight of the hydration, wherein nutritive or artificial sweetener is stevia. In one aspect, the hydration composition comprises at least one amino acid selected from glutamine, L-glutamine, alanine, L-alanine and/or combinations thereof, and when present, the total amount of amino acids included in the hydration composition is about 5% to about 15%, or about 10% to about 30%, or about 15% to about 30%, or about 20% to about 30%, or about 25% to about 30%, based on the total weight of the hydration composition. In one aspect, the hydration composition further comprises a processing aid in an amount of about 0.01% to about 5%, or about 0.01% to about 2%, or about 0.1% to about 5%, or about 0.1% to about 3%, or about 0.1% to about 2%, or about 0.1% to about 1.5%, or about 0.5% to about 1.5%, based on the total weight of the hydration composition, including any amounts or ranges subsumed therein. In one aspect, at least one additional agent is included in the hydration composition selected from vitamins, minerals, caffeine, proteins, herbal/botanical/plant supplements, nutraceuticals, enzymes, probiotics, prebiotics, postbiotics, fermented ingredients, polysaccharides, fats, phytosterols, soluble fiber, insoluble fiber, antioxidants, glucosamine, glycosaminoglycans, adaptogens, nootropics, hormones, flavoring, colorants, preservatives, anti-caking agents, gelling agents, antimicrobials, citric acid and/or other acidifiers, emulsifiers, effervescing agents, and/or excipients.

In one aspect, the hydration composition as described herein, in the form of a powder, gel, emulsion, or effervescent tablet, having about 0% to about 1%, or about 0.005% to about 0.5%, or about 0.01% to about 0.1% by weight water, includes (a) allulose in an amount of about 20% to about 40%, or about 25% to about 35%, or about 28% to about 32%, based on the total weight of the hydration composition; (b) one or more sodium salts, wherein the one or more sodium salts is sodium chloride, sodium citrate or a mixture or combination thereof in an amount of about 7% to about 15%, or about 7% to about 12%, or about 8% to about 12%, or about 9% to about 12%, based on the total weight of the hydration composition; (c) one or more potassium salts, wherein the one or more potassium salts is potassium citrate, dipotassium phosphate or mixtures thereof in an amount of about 3% to about 10%, or about 3% to about 8%, or about 5% to about 12%, or about 5% to about 10%, or about 5% to about 8%, based on the total weight of the hydration composition. In one aspect, the amount of a nutritive or artificial sweetener is about 0.01% to about 3%, or about 0.01% to about 2%, or about 0.01% to about 1.5%, or about 0.01% to about 1.2%, or about 0.01% to about 1.1% or about 0.01% to about 1%, or 0% based on the total weight of the hydration, wherein nutritive or artificial sweetener is stevia. In one aspect, the hydration composition comprises at least one amino acid selected from glutamine, L-glutamine, alanine, L-alanine and/or combinations thereof, and when present, the total amount of amino acids included in the hydration composition is about 5% to about 15%, or about 10% to about 30%, or about 15% to about 30%, or about 20% to about 30%, or about 25% to about 30%, based on the total weight of the hydration composition. In one aspect, the hydration composition further comprises a processing aid in an amount of about 0.01% to about 5%, or about 0.01% to about 2%, or about 0.1% to about 5%, or about 0.1% to about 3%, or about 0.1% to about 2%, or about 0.1% to about 1.5%, or about 0.5% to about 1.5%, based on the total weight of the hydration composition, including any amounts or ranges subsumed therein. In one aspect, at least one additional agent is included in the hydration composition selected from vitamins, minerals, caffeine, proteins, herbal/botanical/plant supplements, nutraceuticals, enzymes, probiotics, prebiotics, postbiotics, fermented ingredients, polysaccharides, fats, phytosterols, soluble fiber, insoluble fiber, antioxidants, glucosamine, glycosaminoglycans, adaptogens, nootropics, hormones, flavoring, colorants, preservatives, anti-caking agents, gelling agents, antimicrobials, citric acid and/or other acidifiers, emulsifiers, effervescing agents, and/or excipients.

In one aspect, the hydration composition as described herein, in the form of a powder, gel, emulsion, or effervescent tablet, having about 0% to about 1%, or about 0.005% to about 0.5%, or about 0.01% to about 0.1% by weight water, and includes (a) a core base comprising allulose and sodium chloride, and (b) an electrolyte base comprising one or more potassium salts, and optionally, one or more sodium salts other than sodium chloride, as described herein. In one aspect, at least one of the one or more sodium salts and/or one or more of the potassium salts is a citrate salt, preferably sodium citrate, disodium citrate, trisodium citrate, potassium citrate and/or mixtures or combinations thereof. The core base is about 45% to about 90%, or about 50% to about 85%, or about 55% to about 80%, or about 60% to about 75%, or about 65% to about 70%, based on the total weight of the hydration composition, including any amounts or ranges subsumed therein. The electrolyte base is about 2% to about 20%, or about 5% to about 18%, or about 7% to about 15%, or about 8% to about 12%, based on the total weight of the hydration composition, including any amounts or ranges subsumed therein. In one aspect, the weight ratio of the core base to the electrolyte base in the hydration composition is about 10:1 to 1:1, or about 8:1 to 4:1, or about 7:1 to about 5:1. In one aspect, the amount of a nutritive or artificial sweetener is about 0.01% to about 3%, or about 0.01% to about 2%, or about 0.01% to about 1.5%, or about 0.01% to about 1.2%, or about 0.01% to about 1.1% or about 0.01% to about 1%, or 0% based on the total weight of the hydration, wherein nutritive or artificial sweetener is stevia. In one aspect, the hydration composition further comprises a processing aid in an amount of about 0.01% to about 5%, or about 0.01% to about 2%, or about 0.1% to about 5%, or about 0.1% to about 3%, or about 0.1% to about 2%, or about 0.1% to about 1.5%, or about 0.5% to about 1.5%, based on the total weight of the hydration composition, including any amounts or ranges subsumed therein. In one aspect, at least one additional agent is included in the hydration composition selected from vitamins, minerals, caffeine, proteins, herbal/botanical/plant supplements, nutraceuticals, enzymes, probiotics, prebiotics, postbiotics, fermented ingredients, polysaccharides, fats, phytosterols, soluble fiber, insoluble fiber, antioxidants, glucosamine, glycosaminoglycans, adaptogens, nootropics, hormones, flavoring, colorants, preservatives, anti-caking agents, gelling agents, antimicrobials, citric acid and/or other acidifiers, emulsifiers, effervescing agents, and/or excipients.

In one aspect, the hydration composition as described herein, in the form of a powder, gel, emulsion, or effervescent tablet, having about 0% to about 1%, or about 0.005% to about 0.5%, or about 0.01% to about 0.1% by weight water, and includes (a) a core base comprising allulose, sodium chloride and at least one amino acid, and (b) an electrolyte base comprising one or more potassium salts, and optionally, one or more sodium salts other than sodium chloride. In one aspect, at least one of the one or more sodium salts and/or one or more of the potassium salts is a citrate salt, preferably sodium citrate, disodium citrate, trisodium citrate, potassium citrate and/or mixtures or combinations thereof. In one aspect, the at least one amino acid is L-alanine and/or L-glutamine. The core base is about 45% to about 90%, or about 50% to about 85%, or about 55% to about 80%, or about 60% to about 75%, or about 60% to about 70%, or about 60% to about 65%, based on the total weight of the composition, including any amounts or ranges subsumed therein. The electrolyte base is about 2% to about 20%, or about 5% to about 18%, or about 7% to about 15%, or about 8% to about 15%, or about 10% to about 13%, based on the total weight of the composition, including any amounts or ranges subsumed therein. In one aspect, the weight ratio of the core base to the electrolyte base is about 10:1 to 1:1, or about 8:1 to 3:1, or about 7:1 to about 4:1. In one aspect, the amount of a nutritive or artificial sweetener is about 0.01% to about 3%, or about 0.01% to about 2%, or about 0.01% to about 1.5%, or about 0.01% to about 1.2%, or about 0.01% to about 1.1%, or about 0.01% to about 1%, or 0% based on the total weight of the hydration, wherein nutritive or artificial sweetener is stevia. In one aspect, the hydration composition further comprises a processing aid in an amount of about 0.01% to about 5%, or about 0.01% to about 2%, or about 0.1% to about 5%, or about 0.1% to about 3%, or about 0.1% to about 2%, or about 0.1% to about 1.5%, or about 0.5% to about 1.5%, based on the total weight of the hydration composition, including any amounts or ranges subsumed therein. In one aspect, at least one additional agent is included in the hydration composition selected from vitamins, minerals, caffeine, proteins, herbal/botanical/plant supplements, nutraceuticals, enzymes, probiotics, prebiotics, postbiotics, fermented ingredients, polysaccharides, fats, phytosterols, soluble fiber, insoluble fiber, antioxidants, glucosamine, glycosaminoglycans, adaptogens, nootropics, hormones, flavoring, colorants, preservatives, anti-caking agents, gelling agents, antimicrobials, citric acid and/or other acidifiers, emulsifiers, effervescing agents, and/or excipients.

### Hydration Formulation

In one aspect, a hydration formulation is provided which includes the hydration composition as described herein, which is combined with a beverage product. A beverage product, as defined herein, is suitable for consumption by drinking such as alcoholic or non-alcoholic beverages (e.g., water, carbonated beverages including carbonated water or soda, coffee, tea, wine, beer, liqueurs, spirits, beverages with fruit-flavoring or juices including lemonades, nectars, fruit and vegetable juices including but not limited to pomegranate juice, beetroot juice, cranberry juice, apple juice, pineapple juice, and/or orange juice, milk including dairy milk and dairy milk alternatives including plant-based milks such as almond milk, coconut milk, oat milk, rice milk or soy milk). In one aspect, the beverage product is water or carbonated water. In one aspect, the hydration composition is added to the beverage product by the consumer, such that hydration composition is mixed with the beverage product, and preferably dissolves in the beverage product creating a hydration formulation. In one aspect, the hydration formulation is sold to the consumer (e.g., ready to drink, with the hydration composition incorporated therein).

In one aspect, the hydration composition as described herein, once diluted to the desired concentration as described herein, and when the optional additional ingredients are added, has a total osmolarity that falls within at least one of the standard or “reduced osmolarity ORS” ranges recommended by UNICEF and WHO (200 to 310 mmol/L and 210 to 260 mmol/L, respectively). See WHO Drug Information Vol. 16, No. 2, 2002, “New formula oral hydration salts,” available online at apps.who.int/medicinedocs/en/d/Js4950e/2.4.html. See also “Oral Hydration Salts (ORS) A New Reduced Osmolarity Formulation,” available online at rehydrate.org/ors/who-unicef-statement.html (May 10, 2002). As used herein, “osmolarity” is the concentration of a solution expressed as the total number of solute particles per liter. Osmolarity may also be expressed in units of milliequivalents per liter (meq/L), where 1 meq is equal to 1 mmol of a solute in a solution. In contrast, “osmolality” is the concentration of a solution expressed as the total number of solute particles per kilogram (mOsm/kg).

In one aspect, allulose is present in the hydration formulation with a weight of about 0.1 g/L to about 6 g/L, or about 1 g/L to about 6 g/L, 2 g/L to about 6 g/L, or about 2 g/L to about 5 g/L, or about 2 g/L to about 4 g/L, or about 2 g/L to about 3 g/L, or about 3 g/L to about 6 g/L, or about 3 g/L to about 5 g/L, including any amounts or ranges subsumed therein.

In another embodiment, the hydration formulation includes from 0.75 to 1.5 wt % allulose based on the total weight of the hydration formulation, including any amount or ranges subsumed therein.

In one aspect, the hydration formulation as described herein, is free of any nutritive or artificial sweetener, as defined herein. In one aspect, nutritive and or artificial sweetener is present in the hydration formulation with a weight of about 0.1 g/L to about 2 g/L, or about 0.1 to about 1.5 g/L, or about 0.1 to about 1.25 g/L, or about 0.1 to about 1 g/L, or about 0.1 to about 0.75 g/L, or about 0.1 to about 0.5 g/L, or about 0.1 to about 0.25 g/L, or about 0.5 g/L to about 2 g/L, about 0.5 g/L to about 1.5 g/L, about 0.5 g/L to about 1.25 g/L, about 0.5 g/L to about 1 g/L, about 0.5 g/L to about 0.75 g/L, including any amounts or ranges subsumed therein.

In one aspect, the total amount of sodium salt(s) present in the hydration formulation is about 0.1 g/L to about 6.5 g/L, or about 0.1 g/L to about 6.3 g/L, or about 0.1 g/L to about 6 g/L, or about 0.1 g/L to about 5.5 g/L, or about 0.1 g/L to about 5 g/L, or about 0.1 g/L to about 4.5 g/L, or about 0.1 g/L to about 4 g/L, or about 0.1 g/L to about 3.5 g/L, or about 0.3 g/L to about 6.4 g/L, or about 0.3 g/L to about 6 g/L, or about 0.3 g/L to about 5.5 g/L, or about 0.3 g/L to about 5 g/L, or about 0.3 g/L to about 4.5 g/L, or about 0.3 g/L to about 4 g/L, or about 0.3 g/L to about 3.5 g/L, or about 0.3 g/L to about 3 g/L, or about 0.3 g/L to about 2.5 g/L, or about 0.3 g/L to about 2 g/L, or about 0.3 g/L to about 1.5 g/L, or about 0.75 g/L to about 6.4 g/L, or about 0.75 g/L to about 6 g/L, or about 0.75 g/L to about 5.5 g/L, or about 0.75 g/L to about 5 g/L, or about 0.75 g/L to about 4.5 g/L, or about 0.75 g/L to about 4 g/L, or about 0.75 g/L to about 3.5 g/L, or about 0.75 g/L to about 3 g/L, or about 0.75 g/L to about 2.5 g/L, or about 1 g/L to about 6.3 g/L, or about 1 g/L to about 6 g/L, or about 1 g/L to about 5.5 g/L, or about 1 g/L to about 5 g/L, or about 1 g/L to about 5.5 g/L, or about 1 g/L to about 5 g/L, or about 1 g/L to about 4.5 g/L, or about 1 g/L to about 4 g/L, or about 1 g/L to about 3.5 g/L, or about 1 g/L to about 3 g/L, or about 1 g/L to about 2.5 g/L, including any amounts or ranges subsumed therein.

In one aspect, sodium chloride is present in the hydration formulation is about 0.2 g/L to about 2.2 g/L, or about 0.2 g/L to about 2 g/L, or about 0.2 g/L to about 1.5 g/L or about 0.2 g/L to about 1 g/L, or about 0.2 g/L to about 0.8 g/L, or about 0.3 g/L to about 2 g/L, or about 0.3 g/L to about 1.5 g/L, or about 0.3 g/L to about 1 g/L, or about 0.3 g/L to about 0.8 g/L, or about 0.3 g/L to about 0.7 g/L, including any amounts or ranges subsumed therein.

In one aspect, sodium citrates, including disodium citrate and/or trisodium citrate, is/are present in the hydration formulation with a weight of about 0.2 g/L to about 2.2 g/L, or about 0.2 g/L to about 2 g/L, about 0.2 g/L to about 1.5 g/L or about 0.2 g/L to about 1 g/L, or about 0.2 g/L to about 0.8 g/L, or about 0.3 g/L to about 2 g/L, or about 0.3 g/L to about 1.5 g/L, or about 0.3 g/L to about 1 g/L, or about 0.3 g/L to about 0.8 g/L, or about 0.3 g/L to about 0.7 g/L, including any amounts or ranges subsumed therein.

In one aspect, the total amount of potassium salt(s) present in the hydration formulation is about 0.3 g/L to about 10 g/L, or about 0.3 g/L to about 6 g/L, or about 0.3 g/L to about 5 g/L, or about 0.3 g/L to about 4 g/L, or about 0.3 g/L to about 3 g/L, or about 0.3 g/L to about 2 g/L, or about 0.3 g/L to about 1 g/L, or about 0.6 g/L to about 6 g/L, or about 0.6 g/L to about 5 g/L, or about 0.6 g/L to about 4 g/L, or about 0.6 g/L to about 3 g/L, or about 0.6 g/L to about 2 g/L, or about 0.6 g/L to about 1 g/L, or about 1 g/L to about 6 g/L, or about 1 g/L to about 5 g/L, or about 1 g/L to about 4 g/L, or about 1 g/L to about 3 g/L, including any amounts or ranges subsumed therein.

In one aspect, potassium citrate is present in the hydration formulation with a weight of about 0.2 g/L to about 2 g/L, or about 0.2 g/L to about 1.5 g/L or about 0.2 g/L to about 1 g/L, or about 0.2 g/L to about 0.8 g/L, or about 0.3 g/L to about 2 g/L, or about 0.3 g/L to about 1.5 g/L, or about 0.3 g/L to about 1 g/L, or about 0.3 g/L to about 0.8 g/L, or about 0.3 g/L to about 0.7 g/L, including any amounts or ranges subsumed therein.

In one aspect, dipotassium phosphate is present in the hydration formulation with a weight of about 0.2 g/L to about 2 g/L, or about 0.2 g/L to about 1.5 g/L or about 0.2 g/L to about 1 g/L, or about 0.2 g/L to about 0.8 g/L, or about 0.3 g/L to about 2 g/L, or about 0.3 g/L to about 1.5 g/L, or about 0.3 g/L to about 1 g/L, or about 0.3 g/L to about 0.8 g/L, or about 0.3 g/L to about 0.7 g/L, including any amounts or ranges subsumed therein.

In one aspect, the total amount of amino acids, as described herein, present in the hydration formulation is about 0.3 g/L to about 4 g/L, or about 0.3 g/L to about 3.5 g/L, or about 0.3 g/L to about 3 g/L, or about 0.3 g/L to about 2.5 g/L, or about 0.3 g/L to about 2 g/L, or about 0.3 g/L to about 1.5 g/L, or about 0.3 g/L to about 1 g/L, including any amount or ranges subsumed therein. In one aspect, glutamine or L-glutamine is present in the hydration formulation with a weight of about 0.3 g/L to about 1 g/L, including any amount or ranges subsumed therein. In one aspect, alanine or L-alanine is present in the hydration beverage with a weight of about 0.3 g/L to about 1 g/L, including any amounts or ranges subsumed therein.

In one aspect, the total amount of additional agents, as described herein, included in the hydration formulation is about 0.1 g/L to about 9 g/L, or about 0.1 g/L to about 8 g/L, 0.1 g/L to about 7 g/L, 0.1 g/L to about 6 g/L, 0.1 g/L to about 5 g/L, 0.1 g/L to about 4 g/L, 0.1 g/L to about 3 g/L, 0.1 g/L to about 2 g/L, 0.1 g/L to about 1 g/L, or about 0.5 g/L to about 5 g/L, or about 0.5 g/L to about 4 g/L, or about 0.5 g/L to about 3 g/L, or about 0.5 g/L to about 2 g/L, or about 0.5 g/L to about 1 g/L, or about 1 g/L to about 5 g/L, or about 1 g/L to about 4 g/L, or about 1 g/L to about 3 g/L, or about 1 g/L to about 2 g/L, including any amounts or ranges subsumed therein. In one aspect, the additional agents include vitamins, preferably vitamin B and vitamin C, more preferably vitamin B3, vitamin B5, vitamin B6, vitamin B12, and vitamin C.

In one aspect, the osmolality of the hydration formulation as described herein is about 210 mOsm/L to about 280 mOsm/L, or about 220 mOsm/L to about 280 mOsm/L, or about 230 mOsm/L to about 280 mOsm/L, or about 240 mOsm/L to about 280 mOsm/L, or about 250 mOsm/L to about 280 mOsm/L, or about 260 mOsm/L to about 280 mOsm/L, 210 mOsm/L to about 270 mOsm/L, or about 215 mOsm/L to about 270 mOsm/L, or about 220 mOsm/L to about 270 mOsm/L, or about 225 mOsm/L to about 270 mOsm/L, or about 230 mOsm/L to about 270 mOsm/L, or about 235 mOsm/L to about 270 mOsm/L, or about 240 mOsm/L to about 270 mOsm/L, or about 245 mOsm/L to about 270 mOsm/L, or about 250 mOsm/L to about 270 mOsm/L, or about 255 mOsm/L to about 270 mOsm/L, or about 260 mOsm/L to about 270 mOsm/L, or about 220 mOsm/L to about 268 mOsm/L, or about 225 mOsm/L to about 268 mOsm/L, or about 230 mOsm/L to about 268 mOsm/L, or about 240 mOsm/L to about 268 mOsm/L, or about 250 mOsm/L to about 268 mOsm/L, including any amounts or ranges subsumed therein. In one aspect, the osmolality of the hydration formulation as described herein is at least about 210 mOsm/L, or at least about 215 mOsm/L, or at least about 220 mOsm/L, or at least about 225 mOsm/L, or at least about 230 mOsm/L, or at least about 235 mOsm/L, or at least about 240 mOsm/L, or at least about 245 mOsm/L, or at least about 250 mOsm/L, or at least about 255 mOsm/L, but not more than about 260 mOsm/L.

In one aspect, the hydration formulation as described herein, includes (a) allulose in an amount of about 0.1 to about 6 g/L, or about 1 g/L to about 6 g/L, or about 3 g/L to about 6 g/L; (b) one or more sodium salts, in an amount of about 1 to about 5 g/L, or about 1 g/L to about 3 g/L, or about 0.3 g/L to about 1.5 g/L; and (c) one or more potassium salts, in an amount of about 0.3 g/L to about 6 g/L, or about 0.3 g/L to about 3 g/L, or about 0.6 g/L to about 6 g/L or about 0.6 g/L to about 3 g/L. In one aspect, the hydration composition has at least one citrate salt selected from sodium citrates, potassium citrate, zinc citrate, and/or any combinations or mixtures thereof. In one aspect, at least one of the one or more sodium salts and/or one or more of the potassium salts is a citrate salt, preferably sodium citrate, disodium citrate, trisodium citrate, potassium citrate and/or mixtures or combinations thereof. In one aspect, there is about 0.1 g/L to about 2 g/L, or about 0.1 g/L to about 1.5 g/L, or about 0.2 g/L to about 1 g/L, or about 0.1 g/L to about 0.5 g/L of nutritive or artificial sweetener, wherein nutritive or artificial sweetener is stevia. The hydration formulation may optionally include about 0.1 g/L to about 9 g/L, or about 0.1 to about 5 g/L, or about 0.1 g/L to about 3 g/L of additional agents, as described herein.

In one aspect, the hydration formulation as described herein, includes (a) allulose in an amount of about 0.1 to about 6 g/L, or about 1 g/L to about 6 g/L, or about 3 g/L to about 6 g/L; (b) sodium salt, wherein the sodium salt is sodium chloride, sodium citrate or a mixture thereof in an amount of about 1 to about 5 g/L, or about 1 g/L to about 3 g/L, or about 0.3 g/L to about 1.5 g/L; (c) potassium salt, wherein the potassium salt is potassium citrate, dipotassium phosphate or mixtures thereof in an amount of about 0.3 g/L to about 6 g/L, or about 0.3 g/L to about 3 g/L, or about 0.6 g/L to about 6 g/L, or about 0.6 g/L to about 3 g/L. In one aspect, there is about 0.1 g/L to about 2 g/L, or about 0.1 g/L to about 1.5 g/L, or about 0.2 g/L to about 1 g/L, or about 0.1 g/L to about 0.5 g/L of nutritive or artificial sweetener, wherein nutritive or artificial sweetener is stevia. The hydration formulation may optionally include about 0.1 g/L to about 9 g/L, or about 0.1 to about 5 g/L, or about 0.1 g/L to about 3 g/L of additional agents, as described herein.

In one aspect, the hydration formulation as described herein, includes (a) allulose in an amount of about 0.1 to about 6 g/L, or about 1 g/L to about 6 g/L, or about 3 g/L to about 6 g/L; (b) sodium salt, wherein the sodium salt is sodium chloride, sodium citrate or a mixture thereof in an amount of about 1 to about 5 g/L, or about 1 g/L to about 3 g/L, or about 0.3 g/L to about 1.5 g/L; (c) potassium salt, wherein the potassium salt is potassium citrate, dipotassium phosphate or mixtures thereof in an amount of about 0.3 g/L to about 6 g/L, or about 0.3 g/L to about 3 g/L, or about 0.6 g/L to about 6 g/L, or about 0.6 g/L to about 3 g/L. In one aspect, there is about 0.1 g/L to about 2 g/L or about 0.1 g/L to about 1.5 g/L, or about 0.2 g/L to about 1 g/L, or about 0.1 g/L to about 0.5 g/L of nutritive or artificial sweetener, wherein nutritive or artificial sweetener is stevia. In one aspect, the hydration composition comprises at least one amino acid, preferably selected from glutamine, L-glutamine, alanine, L-alanine and/or combinations thereof, and when present, the total amount of amino acids included in the hydration composition is about 0.6 to about 2 g/L. The hydration formulation may optionally include about 0.1 g/L to about 9 g/L, or about 0.1 to about 5 g/L, or about 0.1 g/L to about 3 g/L of additional agents, as described herein.

In one aspect, the hydration formulation as described herein, includes (a) a core base comprising allulose and sodium chloride, and (b) an electrolyte base comprising one or more potassium salts, and optionally, one or more sodium salts other than sodium chloride. In one aspect, at least one of the one or more sodium salts and/or one or more of the potassium salts is a citrate salt, preferably sodium citrate, disodium citrate, trisodium citrate, potassium citrate and/or mixtures or combinations thereof. In one aspect, the core base is about 3 to about 7 g/L, and the electrolyte base is about 0.2 to about 2 g/L. In one aspect, there is about 0.1 g/L to about 2 g/L, or about 0.1 g/L to about 1.5 g/L, or about 0.2 g/L to about 1 g/L, or about 0.1 g/L to about 0.5 g/L of nutritive or artificial sweetener, wherein nutritive or artificial sweetener is stevia. In one aspect, there is about 0.1 to about 0.2 g/L of a processing aid. The hydration formulation may optionally include about 0.1 g/L to about 9 g/L, or about 0.1 to about 5 g/L, or about 0.1 g/L to about 3 g/L of additional agents, as described herein.

In one aspect, the hydration formulation as described herein, includes a (a) core base comprising allulose, sodium chloride and at least one amino acid, and (b) an electrolyte base comprising one or more potassium salts, and optionally, one or more sodium salts other than sodium chloride. In one aspect, at least one of the one or more sodium salts and/or one or more of the potassium salts is a citrate salt, preferably sodium citrate, disodium citrate, trisodium citrate, potassium citrate and/or mixtures or combinations thereof. In one aspect, the at least one amino acid is L-alanine and/or L-glutamine. In one aspect, the core base is about 3 to about 6 g/L, and the electrolyte base is about 0.1 to about 1 g/L. In one aspect, there is about 0.1 g/L to about 2 g/L or about 0.1 g/L to about 1.5 g/L, or about 0.2 g/L to about 1 g/L, or about 0.1 g/L to about 0.5 g/L of nutritive or artificial sweetener, wherein nutritive or artificial sweetener is stevia. In one aspect, there is about 0.1 to about 0.07 g/L of a processing aid. The hydration formulation may optionally include about 0.1 g/L to about 9 g/L, or about 0.1 to about 5 g/L, or about 0.1 g/L to about 3 g/L of additional agents, as described herein.

In one aspect, the hydration formulation as described herein, has a beverage hydration index (BHI) of greater than 1. In one aspect, the hydration formulation as described herein, has a BHI of 1.1 to 2.0, or 1.1 to 1.6, or 1.1 to 1.5, or 1.1 to 1.4, or 1.2 to 2.0, or 1.2 to 1.6, or 1.2 to 1.5, or 1.2 to 1.4, or 1.2 to 1.3, or 1.3 to 2.0, or 1.3 to 1.6, or 1.3 to 1.5, or 1.4 to 2.0, or 1.4 to 1.6, or 1.4 to 1.5, or 1.5 to 2.0, or 1.5 to 1.6, including any ranges subsumed therein. BHI is a composite measure of fluid balance after consuming a beverage relative to water, and may be calculated as the ratio of cumulative urine mass for distilled water divided by the cumulative urine mass of the test beverage at certain time points (see, e.g., Maughan et al., “A randomized trial to assess the potential of different beverages to affect hydration status: Development of a beverage hydration index”, The American Journal of Clinical Nutrition, 103(3), 717-723 (2016)). A BHI over 1 means more water is retained in the body than if the drinker had consumed an equal volume of still water.

### Plasma Osmolality

Plasma osmolality is a test that assesses electrolyte-water balances. It measures the concentration of dissolved particles, e.g., osmolality, in blood. This test has been used to help diagnose fluid or electrolyte imbalances and dehydration (Nose et al., “Role of osmolality and plasma volume during rehydration in humans”, Journal of Applied Physiology, 65(1), pp. 325-331 (1988)). Lower plasma osmolality values suggest higher levels of hydration.

In one aspect, plasma osmolality may be tested using a single sample osmometer. The instrument should be calibrated prior to measurements using a set of standard linearity controls encompassing a range of 100-2,000 mOsm/kg. Measurements can be taken in a repeating pattern. Blood samples may be obtained from an indwelling catheter in a vacutainer, or from finger stick if blood cannot be obtained from the catheter.

In one aspect, blood samples for the assessment of plasma osmolality can be collected prior to a set time of activity such as running, jogging, walking, cycling, swimming, rowing, weight training, yoga, or other forms of exercise or physical activity (e.g., about 1 to about 120 minutes, or about 30 to about 100 minutes, or about 50 to about 80 minutes), as well as for a period post-activity, such as at 0 minutes, 15 minutes, 30 minutes, 45 minutes, 60 minutes, and/or 120, preferably where the post-activity period is in the absence of the activity (e.g., resting, or seated rest). In one aspect, administration of the hydration composition and/or formulation as described herein, during or after an activity, may decrease plasma osmolality following the administration of the hydration composition and/or formulation by about 2% to about 9%, or about 3% to about 9%, or about 3% to about 7%, or about 3% to about 6%, or about 3% to about 5%, or about 4% to about 9%, or about 4% to about 8%, about 4% to about 7%, or about 4% to about 6%, about 4% to about 5%, or about 5% to about 9%, or about 5% to about 8%, or about 5% to about 7%, or about 6% to about 9%, or about 6% to about 8%.

### Plasma Volume

Blood plasma is a liquid component of blood that contains proteins and other ingredients of whole blood but lacks blood cells. It accounts for approximately 55% of the total blood volume in the body. It is mostly water (up to 95% by volume) and contains dissolved proteins, glucose, clotting factors, electrolytes, hormones, carbon dioxide, oxygen, and excretory products. It is essential in an intravascular osmotic effect that maintains electrolyte balance. A high plasma volume can improve performance by increasing stroke volume and maximal cardiac output, as well as improving muscle perfusion. Plasma volume expansion improves thermoregulatory responses to exercise by boosting cutaneous blood flow. Higher plasma volumes suggest higher levels of hydration (see, e.g., N. Fellmann, “Hormonal and plasma volume alterations following endurance exercise. A brief review”, Sports Med, 13(1), pp. 37-49 (1992)).

In one aspect, blood samples for the assessment of plasma volume can be collected prior to a set time of activity such as running, jogging, walking, cycling, swimming, rowing, weight training, yoga, or other forms of exercise or physical activity (e.g., about 1 to about 120 minutes, or about 30 to about 100 minutes, or about 50 to about 80 minutes), as well as for a period post-activity, such as at 0 minutes, 15 minutes, 30 minutes, 45 minutes, 60 minutes, and/or 120, preferably where the post-activity period is in the absence of the activity (e.g., resting, or seated rest). In one aspect, it is possible to use the Dill and Costil equation of hemoglobin and hematocrit to calculate the percent plasma volume change. Both hemoglobin and hematocrit can be determined using a Hemapoint© with only 8 microliters of whole blood via vacutainer or finger stick, or by obtaining a blood draw from the subject.

In one aspect, administration of the hydration composition and/or formulation as described herein, before, during or after an activity, may increase plasma volume following the administration of the hydration composition and/or formulation by about 2% to about 12%, or about 3% to about 12%, or about 3% to about 10%, or about 3% to about 8%, or about 3% to about 7%, or about 3% to about 6%, or about 4% to about 12%, or about 4% to about 10%, about 4% to about 8%, or about 4% to about 7%, about 4% to about 6%, or about 5% to about 12%, or about 5% to about 10%, or about 5% to about 8%, or about 6% to about 12%, or about 6% to about 10%, or about 6% to about 8%.

### Time to Exhaustion Trial

In one aspect, administration of the hydration composition and/or formulation as described herein, before or during an activity designed to continue until volitional exhaustion, may increase the time to reach volitional exhaustion following the administration of the hydration composition. As used herein, volitional exhaustion is a subject's inability to continue a physical activity (e.g., exercise) despite any external encouragement to continue said physical activity.

The hydration composition, as described herein, may be used in an edible product. An edible product, as defined herein, is suitable for being eaten or otherwise consumed such as gummies or frozen treats including popsicles, slush, shaved ice, sorbets, ice creams.

In one aspect, provided is a method for making the hydration composition as disclosed herein. In one aspect, a first batch is mixed in a blender for about 1 minutes to about 60 minutes, or about 5 minutes to about 30 minutes, wherein the first batch comprises the allulose, one or more sodium salt, and one or more potassium salts, and optionally an acidifier and/or an amino acid; a second batch, is prepared wherein the second batch comprises a processing aid, and, optionally, a nutritive or artificial sweetener and/or at least one additional agent, preferably one or more vitamins and/or minerals; and the first and the second batches are combined and mixed for about 1 minute to about 60 minutes, or about 5 minutes to about 30 minutes, depending on the batch size.

In one aspect, provided is a method for making the hydration composition as disclosed herein. In one aspect, a first batch is combined and mixed in a ribbon blender for about 1 minutes to about 60 minutes, or about 5 minutes to about 30 minutes, wherein the first batch comprises the allulose, one or more sodium salt(s), preferably sodium chloride and at least one additional sodium salt other than sodium chloride such as sodium citrate, and one or more potassium salt(s), preferably potassium citrate and/or dipotassium phosphate. This first batch may also include, an acidifier such as citric acid, and optionally, at least one amino acid, where the amino acid may be L-alanine and/or L-glutamine. Following the mixing step, a portion of the first batch, preferably, about half by weight, is retained in the ribbon blender. Separate from the first batch, a second batch is prepared a processing aid, and, optionally, a nutritive or artificial sweetener and/or at least one additional agent such as vitamin(s) and/or mineral. The second batch is then added along the center axle of the ribbon blender. Then, the remaining portion of the first batch is added to the ribbon blender which is mixed for about 1 minute to about 60 minutes, or about 5 minutes to about 30 minutes, depending on the batch size.

In one aspect, provided in a method for hydrating a subject in need thereof, by orally administering the hydration formulation as described herein. As used herein, a subject in need thereof is preferably a human, who may be in need of hydration. The rehydration formulation is prepared by diluting and dissolving a rehydration composition as described herein, in a beverage product so that it is dissolved, and the hydration formula has an osmolarity as described herein. The hydration formulation is then consumed or ingested by the subject in need thereof.

The following Examples are provided to further illustrate an understanding of the invention. The Examples are not intended to limit the scope of the claims.

### EXAMPLES

### Example 1

The hydration composition as described herein is made by combining allulose, citric acid, flavorings, sodium citrate, potassium citrate, dipotassium phosphate sodium chloride, and optionally, amino acids (e.g., L-glutamine and/or L-alanine) in a first batch. The first batch is mixed in a ribbon blender for a predetermined mixing time (about 5-30 minutes) depending on the batch size, rotations per minute and design of the blending system. A portion of first batch (preferably, about half by weight) is retained in the ribbon blender. Separate from the first batch, a second batch is made which includes a combination of the vitamins, silicon dioxide and stevia. This batch is then added along the center axle of the ribbon blender. Then, the remaining portion of the first batch is added back to the ribbon blender followed by mixing for 5-30 minutes depending on the batch size, rotations per minute and design of the blending system.

### Example 2

The compositions in the Table below were prepared in accordance with the method set forth in Example 1 hereinabove.

| | Hydration | Hydration |
|---|---|---|
| | Comp. A | Comp. B |
| | (wt %) | (wt %) |
| Allulose | 62.22 | 30.78 |
| Sodium Salt (trisodium citrate and sodium | 9.72 | 11.2 |
| chloride) | | |
| Potassium Salt (potassium citrate and | 6.4 | 7.37 |
| dipotassium phosphate) | | |
| Processing Aid (silicon dioxide) | 0.98 | 1.13 |
| Amino Acids (L-glutamine and L-alanine) | — | 25.7 |
| Vitamins (B3, B5, B6, B12, C) | 0.81 | 0.94 |
| Stevia | 0.79 | 0.91 |
| Flavoring | 19.08 | 21.97 |
| Total | 100 | 100 |

### Example 3

The following exemplifies a hydration formulation as described herein.

| | Hydration Formulation 1 |
|---|---|
| | (g/L) |
| Core Base (Allulose and sodium | 3-7 |
| chloride) | |
| Electrolyte Base (trisodium citrate, | 0.2-2 |
| potassium citrate, and dipotassium | |
| phosphate) | |
| Processing Aid (silicon dioxide) | 0.1-0.2 |
| Vitamins (B3, B5, B6, B12, C) | 0.008-0.8 |
| Stevia | 0.15-0.25 |
| Flavoring | 1-1.75 |

About 13-16 g of Hydration Composition A is dissolved in about 0.473 L of water. The osmolarity of hydration formulation 1 is about 240 to about 255 mmol/L.

### Example 4

The following exemplifies a hydration formulation as described herein.

| | Hydration Formulation 2 |
|---|---|
| | (g/L) |
| Core Base (Allulose, sodium chloride and | 3-6 |
| L-glutamine and L-alanine) | |
| Electrolyte Base (trisodium citrate, | 0.1-1 |
| potassium citrate, and dipotassium | |
| phosphate) | |
| Processing Aid (silicon dioxide) | 0.07-0.1 |
| Vitamins (B3, B5, B6, B12, C) | 0.007-0.7 |
| Stevia | 0.15-0.25 |
| Flavoring | 1-1.75 |

About 13-16 g of Hydration Composition B is dissolved in about 0.473 L of water. The osmolarity of hydration formulation 2 is about 240 to about 255 mmol/L.

### Example 5

### Water Absorption Across Caco-2/TC7 Monolayers

In vitro experiments were conducted to measure the effect of an allulose-containing hydration beverage with limited amounts of stevia and otherwise free of nutritive, non-nutritive and artificial sweeteners on liquid absorption across the in vitro intestinal barrier using mature Caco-2/TC7 epithelial model.

Caco-2 cells, originated from a well-differentiated human colon adenocarcinoma, is an in vitro epithelial model widely used to study intestinal absorption. The epithelial monolayer exhibits the characteristic functions of small intestinal enterocytes, including organization of the apical brush border, high expression of key transporters (e.g., P-gp and BCRP) and intercellular tight junctions (e.g., ZO-1 and occludin). Such features result in an epithelial monolayer with very low permeability, similar to the intestinal barrier in vivo, providing an ideal in vitro model for studying intestinal absorption.

### Reference Compounds and Test Items

| | Negative Controls | Positive Controls |
|---|---|---|
| Reference Substance | Medium | Mannitol |
| Supplier | Sigma-Aldrich | Sigma-Aldrich |
| CAS Number | NA | 69-65-8 |
| Reference | D1145 supplemented with | M4125 |
| | P4333, N4762, and M7145 | |
| Storage | 2-8 °C | Room temperature |

### Test Item

| | |
|---|---|
| Test | rehydration beverage containing allulose, small amounts of |
| Item (SF) | stevia but otherwise no nutritive, non-nutritive or artificial |
| | sweeteners (SF) |
| Form | Solid - Powder |
| Nature | Final product |
| Main | Trisodium Citrate Dihydrate (Sucroal, Fine Granular |
| Composition | 17401) |
| | Potassium Citrate FCC/USP (Tripotassium) Pwd (33% K) |
| | (H)Dipotassium Phosphate (44.89% K/17.79% P) |
| | Sodium Chloride (Morton Salt, F113820000H) |
| | (H) Ascorbic Acid USP Vitamin C Fine Pwd |
| | (99.5% DW) * |
| | (H)Niacinamide B3 USP Pwd (98.5% DW) |
| | (H)d-Calcium Pantothenate B5 Pwd (85.57% B5, 7.5% Ca) |
| | (H)Pyridoxine HCl (Vitamin B6) USP Pwd (80.21% B6) |
| | Vitamin B12 Cyanocobalamin 1% Pwd USP (0.96% B12) |
| | (H)Syloid Silicon Dioxide 244 540 lbs/pallet |
| | Allulose - AllSweet Allulose Crystalline Powder - AG995 |
| | L-Glutamine |
| | L-Alanine |
| | Citric Acid |
| | Organic Stevia (98% Reb A) 120 mg |
| | Lime Flavor WONF |
| | Lemon Flavor WONF |
| Storage | Room temperature |
| Conditions | |
| Sent | 30 sticks of 13 g each |
| Quantity | |

### Test System

Caco-2/TC7 comprises a homogeneous population of epithelial cells derived from the classical Caco-2 cells. The cells are seeded in polycarbonate filters, which allows them to attach and differentiate to form a tight epithelial monolayer similar to the intestinal barrier. The cells are cultivated for 21 days to reach confluence and maturate, being ready for use from 21 until 31 days after seeding. The test system used in the study is validated by measuring the transepithelial electrical resistance (TEER); only Caco-2/TC7 monolayers that meet the acceptance criteria (TEER >500 ohms) were used for the study.

### Materials

| | | | CAS | | |
|---|---|---|---|---|---|
| Chemicals | Supplier | Reference | Number | Storage | |
| DMEM | Sigma- | D6429 | NA | 2-8 | °C |
| | Aldrich | | | | |
| DMEM - Phenol Red | Sigma- | D1145 | NA | 2-8 | °C |
| Free | Aldrich | | | | |
| Calf Serum | Sigma- | N4762 | NA | −20 | °C |
| | Aldrich | | | | |
| Non-Essential Amino | Sigma- | M7145 | NA | 2-8 | °C |
| Acids | Aldrich | | | | |
| Penicillin- | Sigma- | P4333 | NA | −20 | °C |
| Streptomycin | Aldrich | | | | |
| PBS | Sigma- | D8537 | NA | Room | |
| | Aldrich | | | temperature | |
| Lucifer Yellow | Sigma- | L0144 | 71208-95-6 | Room | |
| | Aldrich | | | temperature | |
| D-Mannitol | Sigma- | M4125 | 69-65-8 | Room | |
| | Aldrich | | | temperature | |
| MTT | Sigma- | M2128 | 298-93-1 | 2-8 | °C |
| | Aldrich | | | | |

| | | | | |
|---|---|---|---|---|
| Trypan Blue Stain | Thermo | 15250 | 72-57-1 | Room |
| | Fisher | | | temperature |

### Apparatus

A Cell incubator was used thermostated at 37±2 °C and 10±1% CO2.

The Plate reader used was from BMG Labtech. A World Precision Instruments Evometer (EVOM2) was used to measure TEER (transepithelial electrical resistance). Calculation software employed was Microsoft Excel 2003, 2007, 2010, 2016 and for Microsoft 365 The Nanodrop spectrophotomer and QuantStudio™ 5 Flex Real-Time were obtained from Thermo-Fisher.

Three concentrations of the test solutions were used: [C1]: 5 mg/ml; [C2]: 10 mg/ml; [C3]: 20 mg/ml. HM was a hydration formula including glucose, and SF (composition indicated above) was a rehydration beverage containing allulose and a limited amount of stevia but otherwise no nutritive, non-nutritive or artificial sweeteners.

### Water Absorption Across the Caco-2/TC7 Monolayer

The water absorption was determined by measuring the volume of liquid (medium) in the apical compartment. An initial volume of 200 μl was added to the apical compartment and changes (gain or loss of volume) to the volume were monitored. Loss of volume means that there is absorption of liquid from apical (top) to basolateral (lower) compartment, while gain of volume means that there is passage of liquid from basolateral to apical compartment. The apical compartment represents the lumen of the intestines while the basolateral compartment represents the membrane between the intestine and the bloodstream.

### Treatment Schedule

For all experiments, the following conditions were tested:
1) Control (vehicle-medium): medium (Assay medium) that was used to solubilize the test items. The medium is Dulbecco's Modified Eagle's Medium-High Glucose supplemented with penicillin, streptomycin, newborn calf serum and MEM non essential amino acid solution.
2) Positive control: Mannitol (2.5%), an osmotic diuretic that elevates osmolality, facilitating the absorption of water. Mannitol was used in the basolateral compartment to mimic the osmolarity of the human body wherein osmolarity is typically lower in the lumen of the digestive system than on the other side of the epithelium (represented in this experiment by the basolateral compartment).
3) rehydration beverages HM (containing glucose) and SF containing allulose and limited amounts of stevia but otherwise no nutritive, non-nutritive or artificial sweeteners at [C1] 5 mg/ml; [C2] 10 mg/ml; [C3] 20 mg/ml.

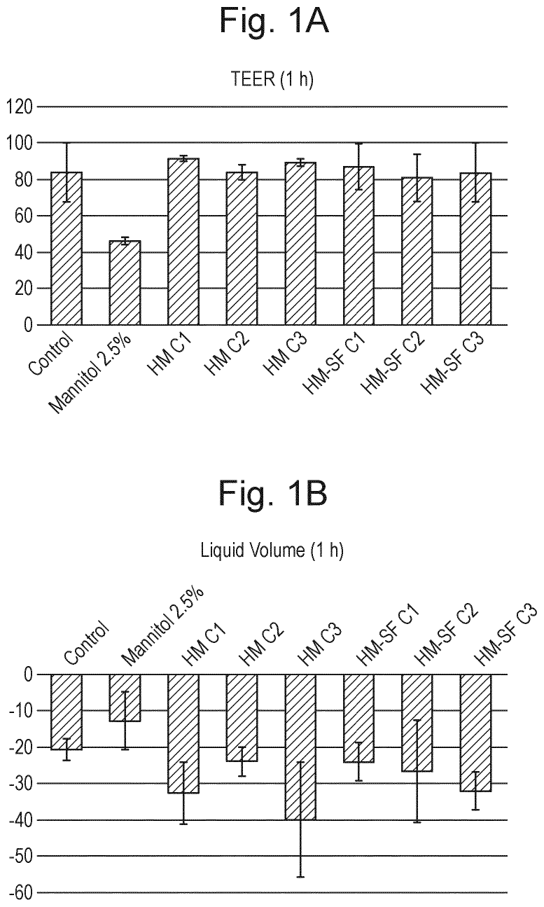

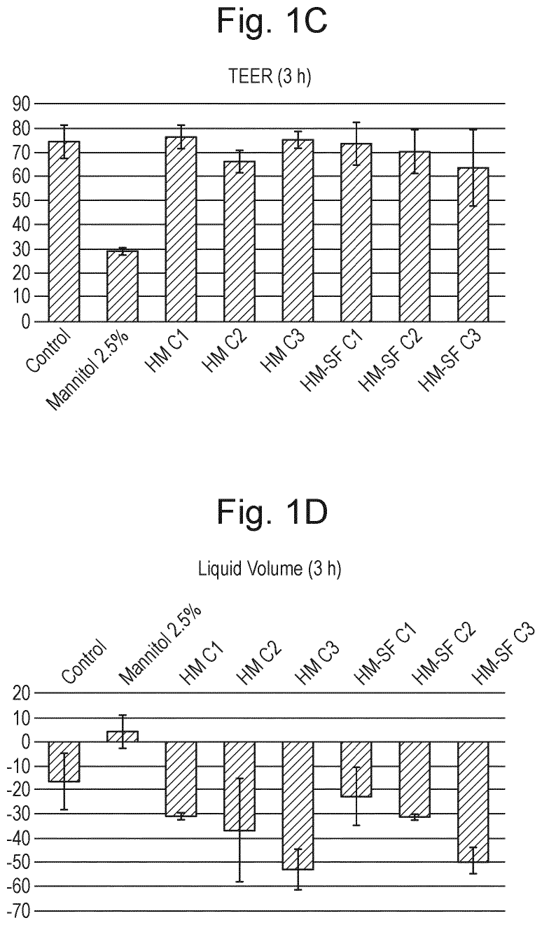

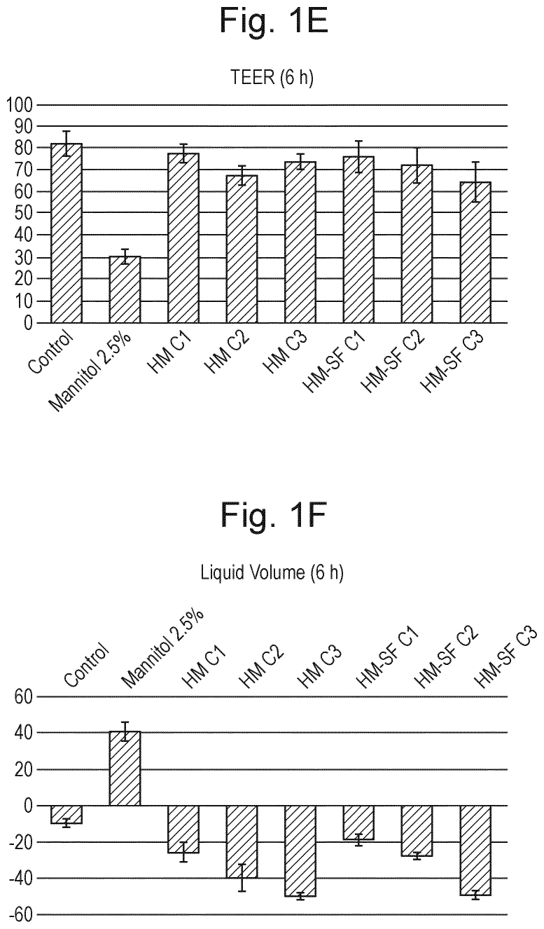

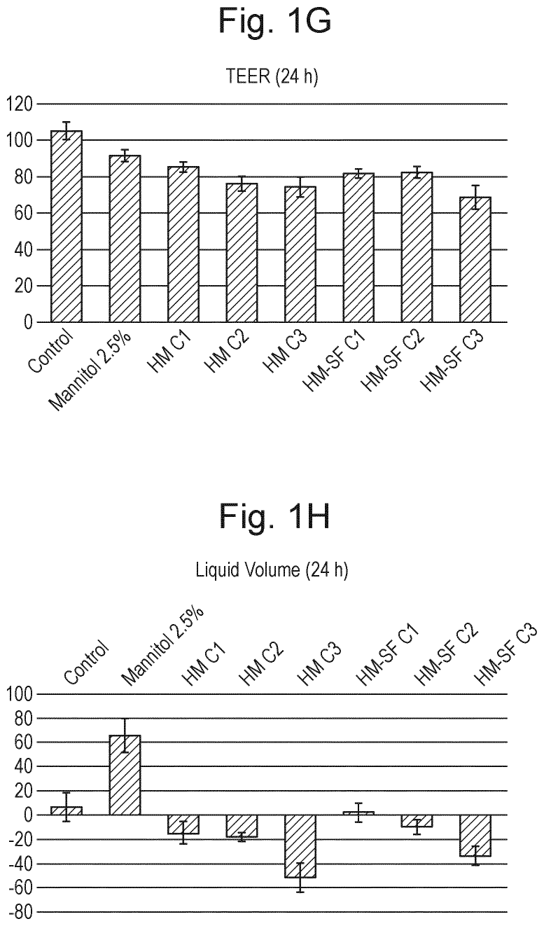

Study A: The treatment was performed in order to check a potential effect of the rehydration beverage containing allulose and limited amounts of stevia but otherwise no nutritive, non-nutritive or artificial sweeteners on liquid absorption across Caco-2/TC7 cells. Cells were treated during 24 hours with the test items in the apical compartment, while normal medium was added in the lower compartment. Control cells received normal medium in both compartments, while the positive control consisted of adding Mannitol 2.5% in the lower compartment to induce water absorption mediated by osmolarity gradient. For this experiment, the volume of medium in the upper compartment and the TEER were measured after 1, 3, 6 and 24 hours of treatment. The cells of this study which did not include mannitol were intended to show that the experimental setup works rather than to mimic osmolarity gradients in humans, as is done in later experiments. The results can be seen in FIG. 1.

Study B: Two distinct treatment schedules were performed.

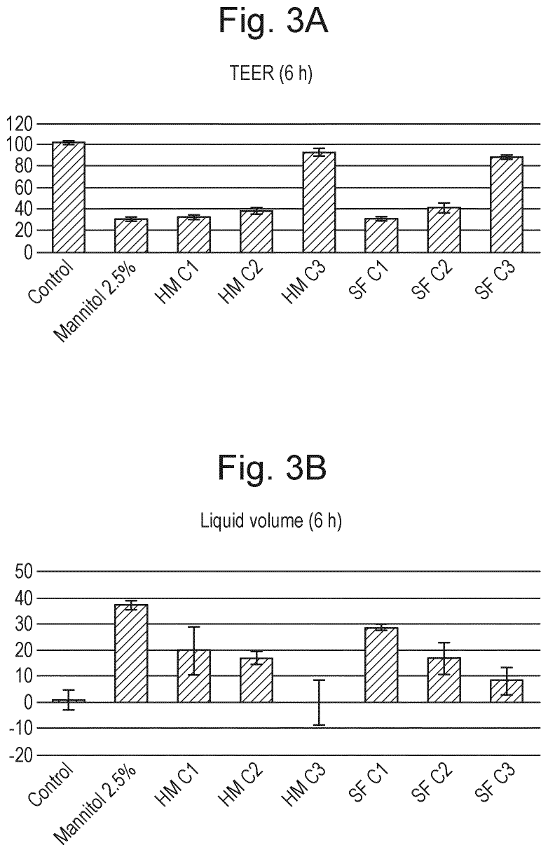

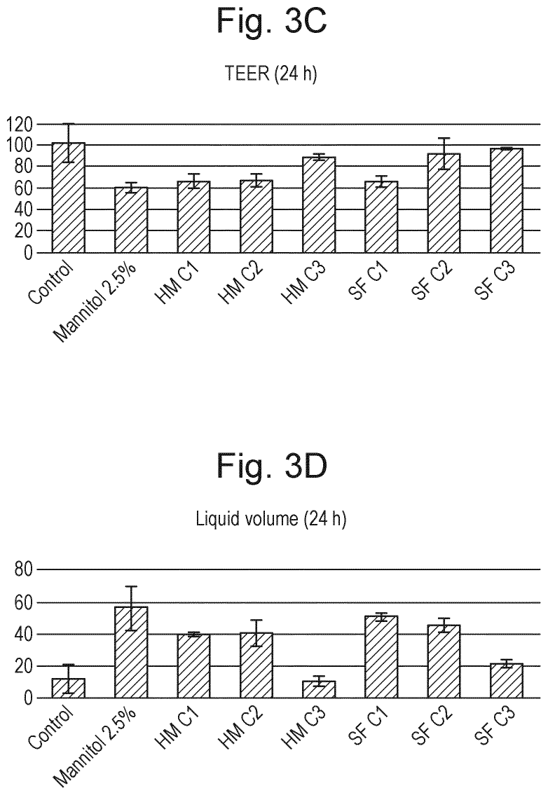

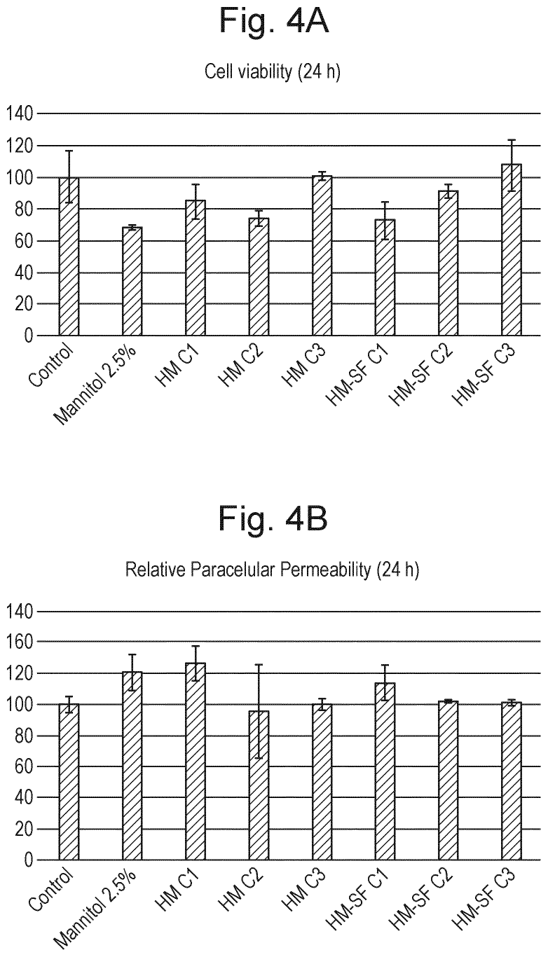

Schedule 1: This treatment schedule was performed in order to check the acute effect of test items in presence of an osmolarity gradient between the apical and basolateral compartment. This approach was tested in order to balance the osmotic gradient between the treatments. Cells were treated during 24 hours with the test items in the apical compartment. Mannitol 2.5% was added in all conditions but the control (just medium in both sides) to equilibrate the osmolarity and facilitate the water absorption. For this experiment, the volume of medium in the upper compartment was measured at 6 and 24 hours, since those time-points showed the best results according to the Study A. In parallel, TEER was also measured at 6 and 24 hours. Results are shown in FIG. 3. At the end, a MTT assay (assay for cellular metabolic activity) and paracellular permeability assessment were performed to assess the cell viability and barrier integrity. Results for 24 hours are shown in FIG. 4.

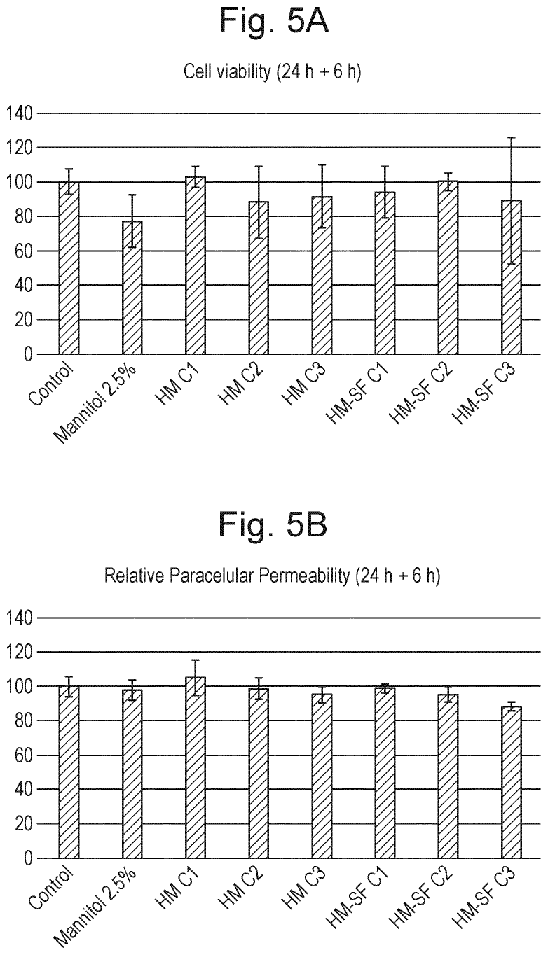

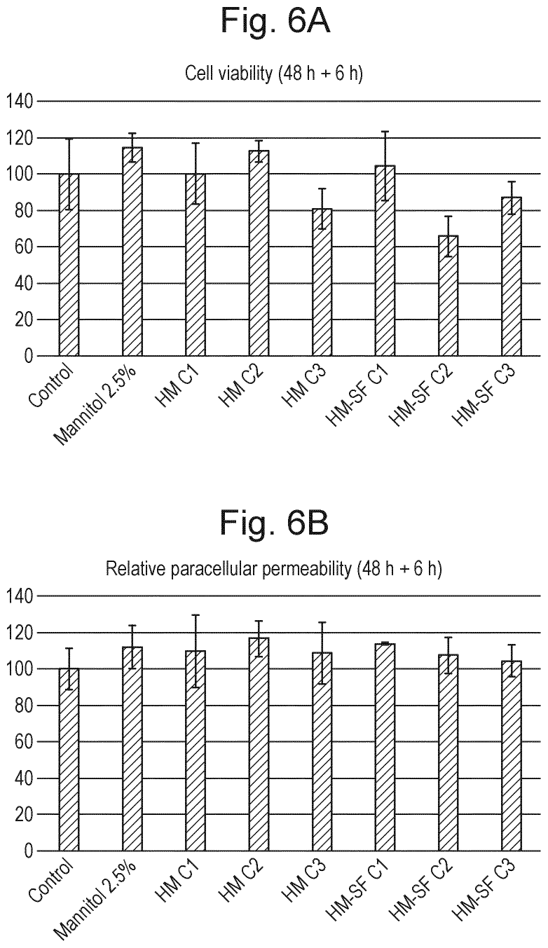

Schedule 2: This treatment schedule was performed in order to check the middle/long-effect of test items, as well as to investigate a potential biological effect on Caco-2/TC7 cells (e.g. regulation of transporter activity) that are independent of osmolarity gradient. Cells were treated during 24 or 48 hours with test items in the apical compartment, while normal medium was added in the lower compartment. Mannitol 2.5% was added in the lower compartment of the Positive Control group, and it was used as an inducer of water absorption mediated by osmolarity gradient. For this experiment, the volume of medium in the upper compartment and the TEER were measured at 24 hours (for 24 h treatment) or 48 hours (for 48 h treatment). Cells were rinsed and mannitol 2.5% was added to the basolateral compartment for all wells (conditions), to stimulate the water passage from luminal to abluminal compartment. After 6 hours, the volume of medium in the apical compartment was measured, in parallel to the measure of TEER. At the end, a MTT assay and paracellular permeability assessment was performed to assess the cell viability and barrier integrity. Results are shown in FIGS. 5 and 6.

### Assessment of Barrier Integrity

The TEER was measured by using an Evometer (EVOM2) to assess the transcellular permeability of the intestinal barrier. At the end of the experiments, cells were rinsed with PBS and submitted to an assay to determine the paracellular permeability. Briefly, LY (200 μM), a marker of paracellular permeability presenting low passage across the intestinal barrier, was added to the apical compartment. Then, filters were placed in 24-well plates containing 600 μl of fresh Assay medium. After one hour of incubation (37 °C, CO2 incubator), the fluorescence emitted by LY was measured in both apical and basolateral compartments.

### Assessment of Cell Viability

Following the permeability assay, cells were rinsed with PBS and the cell viability was determined by the MTT assay.

At the end of the incubation period, the Caco-2/TC7 monolayers were dried with paper towels, and then transferred to another 24-wells plate containing 300 μl/well of MTT dye solution (1 mg/ml MTT in the medium), and the plates were incubated in the dark for 1 hour±5 minutes (37±1 °C, 5±1% CO2).

After incubation in MTT, the Caco-2/TC7 monolayers were dried with paper towels and transferred into a 24-wells plate containing 1 ml of isopropanol, then 1 ml of isopropanol were added to the top of each insert. The plates were incubated for at least 30 minutes at room temperature.

Finally, the inserts were pierced to ensure the mixture recovery of the supernatant and the subnatant isopropanol. Isopropanol extracts were briefly homogenized, transferred onto a 96 wells plate (2 separate aliquots of 200 μl per epidermis insert), and the optical density (OD) was measured at 570 nm in duplicate.

### Results

FIG. 1 represents the first experiments with the test item SF (rehydration beverage containing allulose and small amounts of stevia but otherwise no nutritive, non-nutritive or artificial sweeteners) and the positive control (mannitol). Such experiments were performed in order to investigate the effects of the SF beverage on liquid absorption across the Caco-2/TC7 model, as well as to optimize the best conditions (e.g. time of exposure) for evaluating the liquid absorption.

As expected, the presence of mannitol (2.5%) in the lower compartment induced liquid absorption from luminal to basolateral compartment. This effect is possibly mediated by the osmolarity gradient created by mannitol, as may be expected in human systems where a gradient typically exists of lower osmolarity in the lumen to higher osmolarity in the blood. The mannitol-induced water absorption was followed by a marked decrease on TEER at the first time points (1, 3, and 6 hours), however it recovered at 24 hours. As for mannitol, this effect was possibly mediated by the osmolarity gradient created by adding the test item. In the absence of an osmolarity gradient created by mannitol in the basolateral compartment, fluid flowed from the basolateral compartment to the luminal (apical) compartment.

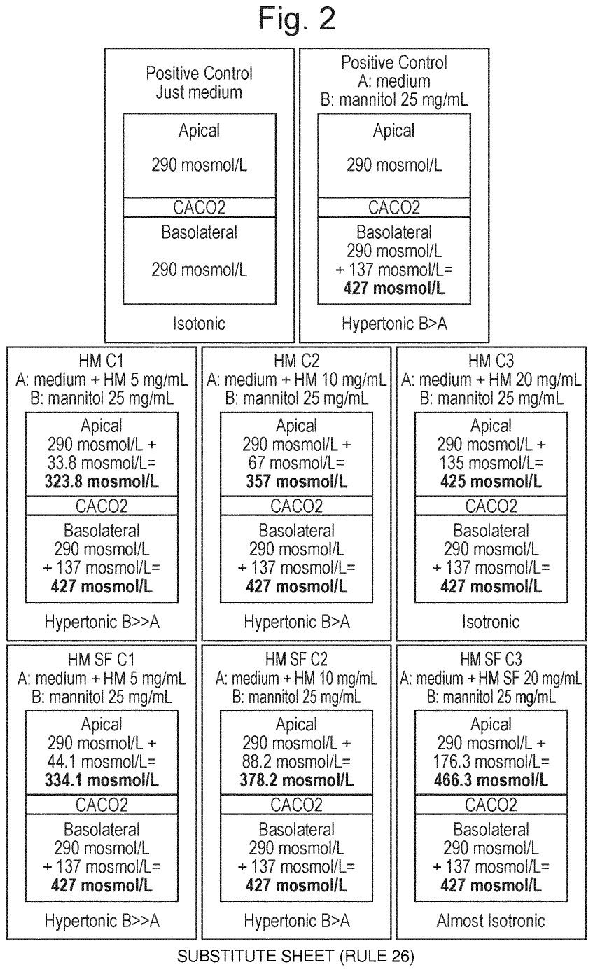

FIG. 2 shows the calculated osmolarity at the luminal and basolateral compartments, according to the described treatments. Adding mannitol in the lower, basolateral, compartment of treated cells wherein the test solution is allulose-containing hydration formula with limited amounts of stevia and otherwise free of nutritive, non-nutritive and artificial sweeteners decreased the difference of osmolarity between the luminal and basolateral compartment, allowing then to observe a positive effect of the test item on liquid absorption. At isotonic conditions (Control and highest concentration of test items [C3]), a small volume of liquid crosses the Caco-2/TC7 cells, without marked effect on TEER. Even though, it is important to highlight that SF test solution induced higher liquid absorption at 6 and 24 hours without any effect on TEER, suggesting it might be mediated by other factors (e.g. active transport) without osmotic gradient. Additionally, when the osmolarity is higher at the basolateral compartment (test items, [C1] and [C2]), test solution (rehydration beverage containing allulose and a small amount of stevia, but otherwise no nutritive, non-nutritive or artificial sweeteners) stimulated liquid absorption. In parallel, the test item at concentrations [C1] and [C2] decreased the TEER, such as the effect observed by Mannitol (FIG. 3). The analysis of cell viability by MTT has shown that treatments having a bigger osmolarity gradient (in particular the mannitol) promoted a slight but consistent reduction on cell viability (20-30% of relative reduction in comparison to control). However, the analysis of paracellular permeability has shown that no treatment had a considerable effect on paracellular barrier integrity, indicating then that the monolayer keeps its integrity after all treatments (FIG. 4) Although a glucose containing hydration formula (HM) behaves somewhat similarly, some consumers can be expected to prefer hydration formulas without nutritive sugar.

A second strategy employed for searching a potential effect of hydration beverage with allulose and limited amounts of stevia but otherwise free of nutritive-, non-nutritive and artificial sweeteners (SF) consisted of treating the cells during 24 and 48 hours, and then exposing the cells to Mannitol (2.5%) in the basolateral compartment during 6 hours (recovery time) to test the hypothesis that the test items could stimulate the liquid absorption at middle/long term. See FIGS. 5 and 6. Referring to FIG. 3, overall, the liquid absorption and TEER values at 6 and 24 hours were similar to the previous experiments (FIG. 1), confirming the positive effect of osmolarity on liquid absorption. The results suggest that the allulose-containing nutritive sweetener-free, artificial sweetener-free, non-nutritive sweetener free-test solution (SF) had an additional effect on mannitol-mediated liquid absorption. FIGS. 3-6. On the other hand, both test items promoted a consistent increase (20-30%) on TEER after 48 hours of treatment, suggesting it could be beneficial for the barrier tightness.

### Discussion

The intestine contains a simple layer of epithelial cells that plays a key role in the uptake of nutrients, reabsorption of water, and the prevention of pathogen invasion. The transport of substances across the epithelial intestine occurs through the transcellular and paracellular pathway. For the transcellular pathway, the substances should be taken up by the epithelial cells by pinocytosis or by action of transporters. The main mechanisms include the passive diffusion across the cell membrane or the carrier-mediated pathway (carrier/receptor-mediated transcellular pathway). For the paracellular pathway, substances should be transported through the lateral borders of the cells. This process depends mainly on the barrier tightness, which is regulated by tight junctions and adherens junctions, proteins that seal the passage between the luminal and basolateral compartments. Caco-2 cells grown on permeable support have been widely used as a tool to study the in vitro absorption of substances in the intestine, since it expresses the main transporters present on intestinal epithelium and presents a well-organized tight junction and adherens junctions at the cell borders. For this reason, the Caco-2 model was chosen to study the effect of the hydration formulation with no nutritive, non-nutritive, or artificial sweeteners on water absorption. The exact mechanism of water movement across the intestinal epithelial barrier is not completely understood, however some evidence has suggested that water absorption in the intestine can occur both through transcellular and paracellular pathways. Another concept that is widely accepted is that the water movement across the epithelium can be coupled to an active ionic transport or driven by an external hydrostatic or osmotic gradient. The results obtained with mannitol have confirmed that the movement of water across the Caco-2/TC7 model is, at least partially, dependent on the presence of an osmotic gradient, since its addition to the basolateral compartment has induced the highest level of liquid absorption. The results obtained with the test items reinforce such hypothesis. For instance, the addition of the hydration formulation with no nutritive, non-nutritive- or artificial-sweeteners test item to the luminal compartment promotes an osmotic gradient of around 130 mosmol/L (for the highest concentration) between the luminal and the basolateral compartments, leading to a dose-dependent movement of water from basolateral to luminal compartment. These results agree with previous study showing that the movement of water is regulated by osmolarity gradient, therefore an increase in the tonicity of the medium modifies the water transport across the membrane and it can regulate the absorption and secretion of water. Similar to the present experiments, this study has also shown that osmotic gradient can decrease the transepithelial electrical resistance, which can also participate in the regulation of water transport. A set of experiments were performed with mannitol added in the lower (basolateral) compartment to balance the osmotic gradient. The results have shown that correcting the osmotic gradient favored the water absorption induced by the test item. For instance, the two lowest concentrations of both test items (C1 and C2), which created an osmotic gradient from basolateral to luminal compartment, induced water absorption to a similar extent observed with mannitol alone. It is noteworthy that this occurs even in presence of lower osmotic gradient when compared to the positive control (Osmotic gradient from basolateral to luminal compartment: Positive Control: 137 mosmol/L;

Hydration Formula with allulose (SF) C2: 49 mosmol/L). On the other hand, when there is no (or very low) osmotic gradient (e.g. Control or highest concentration of the test item), the water absorption is reduced. Interestingly, the highest concentration of Hydration formula with allulose (SF) [C3] induced the water absorption from luminal to basolateral compartment even in presence of higher osmolarity in the luminal compartment (luminal: 466 mosmol/L vs basolateral: 427 mosmol/L). This result suggests that the SF test item might be capable of transporting water against the osmotic gradient. Another set of experiments were performed after middle/long term exposure to the test items, followed by a recovery of 6 hours in presence of mannitol in the lower compartment, to investigate a potential effect of SF on mannitol-mediated water absorption. SF increased the TEER values after 48 hours, suggesting it might increase the barrier tightness, which could be an additional beneficial effect of the allulose-containing hydration formula without nutritive sugar, non-nutritive sweetener or artificial sweetener (SF)

The present study has confirmed that the Caco-2/TC7 is suitable for studying water absorption in vitro. Overall, the allulose containing hydration formula without nutritive sugar non-nutritive sugar or artificial sweetener (SF) had no toxic effects, even at the highest concentrations employed in this study. Other promising findings include the water absorption against the osmotic gradient induced by the (SF) at the highest concentration and the effect on barrier tightness. SF was shown to promote intestinal water absorption. Concentrations of allulose in the hydration formula (based on the total weight of the hydration formula) at which transport across the epithelium may be expected include 5 mg/mL-50 mg/mL, especially 5 mg/mL-40 mg/mL.

### Example 6 (Prophetic)

### Water Absorption Across Caco-2/TC7 Monolayers

Generalized method: Caco-2/TC7 comprises a similar population of intestinal cells derived from the classical Caco-2 cells. The cells are cultivated for 21 days to reach confluence and maturate, being then ready for use. The test system used in the study is the transepithelial electrical resistance (TEER).

The water absorption is determined by measuring the volume of liquid in the apical compartment. An initial volume of 200 μl is added in the apical compartment and changes (gain or loss of volume) on the volume were monitored. Loss of volume means that there is absorption of liquid from apical (top) to basolateral (lower) compartment, while gain of volume means that there is passage of liquid from basolateral to the apical compartment.

For all experiments, the following conditions are tested:
1) Control (vehicle-medium): medium (Assay medium) that is used to solubilize the test items.
2) Positive control: Mannitol (2.5%), an osmotic diuretic that elevates osmolality, facilitating the absorption of water.
3) rehydration beverage containing allulose, small amounts of stevia but otherwise no nutritive, non-nutritive or artificial sweeteners (SF) at [C1] 5 mg/ml; [C2] 10 mg/ml; [C3] 20 mg/ml.

Schedule 1: This treatment schedule is performed in order to check the acute effect of test items in presence of an osmolarity gradient between the apical and basolateral compartment. This approach is tested in order to balance the osmotic gradient between the treatments. Cells are treated during 24 hours with the test items in the apical compartment. Mannitol 2.5% is added in all conditions but the control to equilibrate the osmolarity and facilitate the water absorption. For this experiment, the volume of medium in the upper compartment is measured at 6 and 24 hours. In parallel, TEER is also measured at 6 and 24 hours. At the end, a MTT assay and paracellular permeability assessment are performed to assess the cell viability and barrier integrity.

Schedule 2: This treatment schedule is performed in order to check the middle/long-effect of test items, as well as to investigate a potential biological effect on Caco-2/TC7 cells that are independent of osmolarity gradient. Cells are treated during 24 or 48 hours with test items in the apical compartment, while normal medium is added in the lower compartment. Mannitol 2.5% is added in the lower compartment of the Positive Control group, and it is used as an inducer of water absorption mediated by osmolarity gradient. For this experiment, the volume of medium in the upper compartment and the TEER are measured at 24 hours or 48 hours. Cells are rinsed and mannitol 2.5% is added on basolateral compartment for all conditions, to stimulate the water passage from luminal to abluminal compartment. After 6 hours, the volume of medium in the apical compartment is measured, in parallel to the measure of TEER. At the end, a MTT assay and paracellular permeability assessment is performed to assess the cell viability and barrier integrity.

### Assessment of Barrier Integrity

The TEER is measured by using an Evometer to assess the transcellular permeability of the intestinal barrier. At the end of the experiments, cells are rinsed with PBS and submitted to an assay to determine the paracellular permeability. Briefly, LY, a marker of paracellular permeability presenting low passage across the intestinal barrier, is added to the apical compartment. Then, filters are placed in 24-well plates containing fresh Assay medium. After one hour of incubation, the fluorescence emitted by LY is measured in both apical and basolateral compartments.

### Assessment of Cell Viability

Following the permeability assay, cells are rinsed with PBS and the cell viability is determined by the MTT assay.

At the end of the incubation period, the Caco-2/TC7 monolayers are dried with paper towels, and then transferred to another 24-wells plate containing MTT dye solution, and the plates are incubated in the dark for 1 hour±5 minutes.

After incubation in MTT, the Caco-2/TC7 monolayers are dried with paper towels and transferred into a 24-wells plate containing isopropanol, then isopropanol is added to the top of each insert. The plates are incubated for at least 30 minutes at room temperature.

Finally, the inserts are pierced to ensure the mixture recovery of the supernatant and the subnatant isopropanol. Isopropanol extracts are briefly homogenized, transferred onto a 96 wells plate and the optical density (OD) is measured in duplicate.

At 6 hours intestinal absorption in the range of 5 through 40 mL and 0.8 through 6.7 mL per hour are obtained.

At 24 hours, intestinal absorption in the range of 10-60 mL or 0.4 through 2.5 mL per hour are obtained.

1. A hydration composition comprising:
allulose;
one or more sodium salts;
one or more potassium salts; and
at least one amino acid selected from glutamine, L-glutamine, alanine, L-alanine, or a combination thereof,
wherein the composition comprises from 0.01% to 1.5% of a nutritive or artificial sweetener; and
wherein at least one of the one or more sodium salts and/or one or more of the potassium salts is a citrate salt.

2. The hydration composition of claim 1, wherein the hydration composition is in the form of a powder, gel, emulsion, or effervescent tablet.

3. The hydration composition of claim 1, wherein the nutritive or artificial sweetener is selected from the group consisting of glucose, fructose, galactose, sucrose, lactose, maltose, dextrose, sucralose, honey, agave, molasses, corn syrup, xylitol, sorbitol, acesulfame potassium, monk fruit, sodium saccharide, saccharin, erythritol, neotame, tagatose, aspartame, and stevia and combinations thereof.

4. The hydration composition of claim 1, wherein the citrate salt is selected from sodium citrate, disodium citrate, trisodium citrate, potassium citrate, or any combination thereof.

5. The hydration composition of claim 1, wherein the hydration composition comprises from about 1 wt % to about 15 wt %, based on the total weight of the hydration composition, of glutamine or L-glutamine, and from about 1 wt % to about 15 wt %, based on the total weight of the hydration composition, of alanine or L-alanine.

6. The hydration composition of claim 1, wherein the one or more sodium salts is selected from the group consisting of sodium chloride, monosodium phosphate, disodium phosphate, trisodium phosphate, sodium citrates, sodium carbonate, sodium bicarbonate, sodium lactate, sodium gluconate, tetrasodium acid pyrophosphate, sodium acid sulfate, sodium hydroxide, and any combination thereof.

7. The hydration composition of claim 1, wherein the one or more potassium salt is selected from the group consisting of potassium chloride, potassium phosphate, dipotassium phosphate, potassium citrate, potassium carbonate, potassium bicarbonate, potassium hydroxide and any combination thereof.

8. The hydration composition of claim 1, wherein a weight ratio of allulose to a total amount of sodium and potassium salts is about 4:1 to 1:1.

9. The hydration composition of claim 1 further comprising at least one additional agent, wherein the additional agent is selected from the group consisting of vitamins, minerals, caffeine, proteins, herbal/botanical/plant supplements, nutraceuticals, probiotics, prebiotics, soluble fiber, insoluble fiber, antioxidants, glucosamine, glycosaminoglycans, adaptogens, nootropics, hormones, flavors, colorants, preservatives, anti-caking agents, gelling agents, antimicrobials, citric acid and other acidifiers, emulsifiers, effervescing agents excipients, and mixtures thereof.

10. A method of preparing a hydration formula comprising adding the hydration composition of claim 1 to a beverage product.

11. A hydration formulation comprising the hydration composition of claim 1.

12. The hydration formulation of claim 11, wherein osmolality of the formulation is about 210 mOsm/L to about 280 mOsm/L.

13. The hydration formulation of claim 11, wherein the hydration formulation has a beverage hydration index of 1.1 to 2.0.

14. A hydration formulation comprising a hydration composition, wherein the hydration composition comprises:
a. a core base, wherein the core base comprises allulose and sodium chloride; and
b. an electrolyte base, wherein the electrolyte base comprises one or more potassium salts, and one or more sodium salt excluding sodium chloride;
wherein the core base comprises at least one amino acid, and
wherein at least one of the one or more sodium salts and/or one or more of the potassium salts is a citrate salt.

15. A method for making the hydration composition of claim 1, comprising:
a. mixing a first batch in a blender for about 1 minutes to about 60 minutes, wherein the first batch comprises the allulose, the one or more sodium salt, and the one or more potassium salts, the at least one amino acid, and, optionally, an acidifier;
b. preparing a second batch, wherein the second batch comprises a processing aid, and at least one additional agent selected from one or more vitamins and/or minerals; and
c. combining the first and the second batches.

16. The hydration composition of claim 1, wherein the hydration composition comprises from 0.01% to 0.5% of a nutritive or artificial sweetener, and wherein the nutritive or artificial sweetener is stevia.

17. The hydration composition of claim 1, wherein the hydration composition comprises from 5 wt % to 20 wt %, based on the total weight of the hydration composition, of the one or more sodium salts.

18. The hydration composition of claim 1, wherein the hydration composition comprises from 1 wt % to 20 wt %, based on the total weight of the hydration composition, of the one or more potassium salt.

19. The hydration composition of claim 8, wherein the weight ratio of allulose to the total amount of sodium and potassium salts is 4:1 to 1.5:1.

20. The hydration composition of claim 9, wherein the vitamins are selected from vitamin A, vitamin B1, vitamin B2, vitamin B3, vitamin B5, vitamin B6, vitamin B7, vitamin B12, vitamin C, vitamin D, vitamin E, vitamin K, or any combinations thereof.
